(19) United States
(12) Patent Application Publication
(10) Pub. No.: US 2023/0101743 A1
(43) Pub. Date: Mar. 30, 2023

(54) SUBSTITUTED TRIAZINE COMPOUNDS AND USES THEREOF

(71) Applicant: MUSC FOUNDATION FOR RESEARCH DEVELOPMENT, Charleston, SC (US)

(72) Inventors: Stephen DUNCAN, Hollywood, SC (US); Juitung LIU, Sumter, SC (US); Patrick M. WOSTER, Charleston, SC (US); Yuri PETERSON, Charleston, SC (US); Steven HOLSHOUSER, Charleston, SC (US); Yu-Lin JIANG, Charleston, SC (US)

(21) Appl. No.: 17/790,798

(22) PCT Filed: Jan. 6, 2021
(86) PCT No.: PCT/US2021/012303

§ 371 Date: Jul. 5, 2022

Related U.S. Application Data
(60) Provisional application No. 62/957,468, filed on Jan. 6, 2020.

(51) Int. Cl. C07D 251/40; C07D 403/14; C07D 403/04

(52) U.S. Cl. CPC C07D 251/40; C07D 403/14; C07D 403/04

(57) ABSTRACT

The present invention relates to compounds of formula (I): including any stereochemically isomeric form thereof, or pharmaceutically acceptable salts thereof, for the treatment of, for example, hypercholesterolemia.

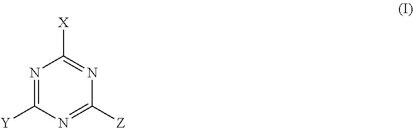

### CROSS-REFERENCE TO RELATED APPLICATIONS

This application claims priority of U.S. Provisional Application No. 62/957,468, filed Jan. 6, 2020, the entire contents of which is hereby incorporated by reference herein.

### STATEMENT REGARDING FEDERALLY SPONSORED RESEARCH OR DEVELOPMENT

This invention was made with Government support under NIH/NHGRI grant U01 HG006398. The Government has certain rights in the invention.

### FIELD OF THE INVENTION

The invention relates generally to substituted triazine compounds useful for the treatment of hypercholesterolemia. All documents cited to or relied upon below are expressly incorporated herein by reference.

### BACKGROUND OF THE INVENTION

Hypercholesterolemia is a key contributor to atherosclerosis and cardiovascular disease. The liver plays an essential role in regulating the serum cholesterol level by producing and recycling low-density lipoprotein (LDL).

Although there are existing treatments such as statins and PCSK9 inhibitors, a subpopulation of patients do not respond efficiently, including patients with homozygous familial hypercholesterolemia (FH). There is a need for alternative treatments for this disease.

### SUMMARY OF THE INVENTION

The present invention is directed to a compound of formula (I):

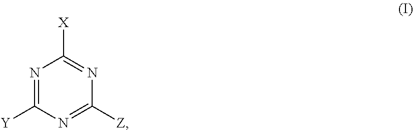

wherein:

X and Y, independently of each other, is halogen, SH, SC(O)CH₃, NH-lower alkyl, N(lower alkyl)₂, NH-alkoxy or NHNH₂;

Z is hydroxy, halogen, heterocycloalkyl or NR¹R²; and

R¹ and R², independently of each other, is hydrogen, alkyl, alkoxy, cycloalkyl, unsubstituted phenyl or phenyl mono-, bi- or tri-substituted independently with NH-phenyl, halogen, hydroxyl, lower alkyl or NH-heteroaryl, wherein said heteroaryl is unsubstituted or mono- or bi-substituted independently with SH or NHCH(CH₃)₂,

or a pharmaceutically acceptable salt thereof.

The present invention is also directed to a pharmaceutical composition, comprising a therapeutically effective amount of a compound according to formula (I), or a pharmaceutically acceptable salt thereof, and a pharmaceutically acceptable carrier.

The present invention is further directed to a method for the treatment of hypercholesterolemia, comprising the step of administering to a patient in need thereof a therapeutically effective amount of a compound according to formula (I), or a pharmaceutically acceptable salt thereof, and a pharmaceutically acceptable carrier.

### BRIEF DESCRIPTION OF THE DRAWINGS

FIG. 1 is a graph showing that DL-1 reduces APOB secretion in the culture medium of hepatic cells.

FIG. 2 shows that DL-1 reduced total cholesterol level in culture medium of hepatic cells.

FIG. 3 shows that DL-1 has no impact on hepatic character.

FIG. 4 shows that DL-1 reduces APOB secretion level in a dose-response manner without affecting albumin.

FIGS. 5 and 6 show that DL-1 reduces APOB levels in a time-dependent manner without affecting albumin and APOA levels.

FIG. 7 shows that DL-1 has no impact on APOB mRNA and intracellular protein levels.

FIG. 8 shows that DL-1 has no impact on hepatic lipid accumulation compared to an MTTP inhibitor.

FIG. 9 demonstrates that DL-1 derivatives show APOB reduction in human iPSC-derived hepatocytes.

FIG. 10 shows that the thiol group helps reduce hepatic APOB secretion.

FIG. 11 shows a dose-response analysis of DL-1 derivatives.

FIG. 12 shows that DL-1 and derivatives induced intracellular APOB accumulation independently of mRNA expression and LDL uptake.

FIG. 13 shows that compounds of the invention induced presecretory intracellular APOB aggregation and lysosomal degradation.

### DETAILED DESCRIPTION OF THE INVENTION

It is to be understood that the descriptions of the present invention have been simplified to illustrate elements that are relevant for a clear understanding of the present invention, while eliminating, for the purpose of clarity, many other elements found in typical pharmaceutical compositions. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

The use of pluripotent stem cell-derived hepatocytes for studying liver disease has become an attractive platform for drug development. The established protocols for differentiating human induced pluripotent stem cells (iPSCs) into hepatocyte-like cells (HLCs) provides new opportunities to study liver disease and metabolism in high-throughput formats. Using human iPSC-derived HLCs, a screen was performed on 10K small molecules from the South Carolina Compound Collection to identify novel molecules which could lower the secreted level of apolipoprotein B (APOB). APOB is the central and essential protein component of (v)LDL particles.

Eleven validated initial hits were identified that significantly reduced APOB levels in the culture medium without affecting cell viability. Moreover, by treating FH patient-derived iPSC-HLCs, five of the identified small molecules significantly decreased secreted APOB level, indicating that the compounds act independently of the LDLR. Of the eleven initial compounds, three appear to have novel mechanisms of action and could represent new classes of cholesterol lowering drugs. Extrapolation from the representative set identified and additional 18 structurally related compounds. After testing, 13 were capable of reducing APOB levels secreted from iPSC-derived hepatocytes.

In one embodiment, the invention is directed to at least the following compounds:

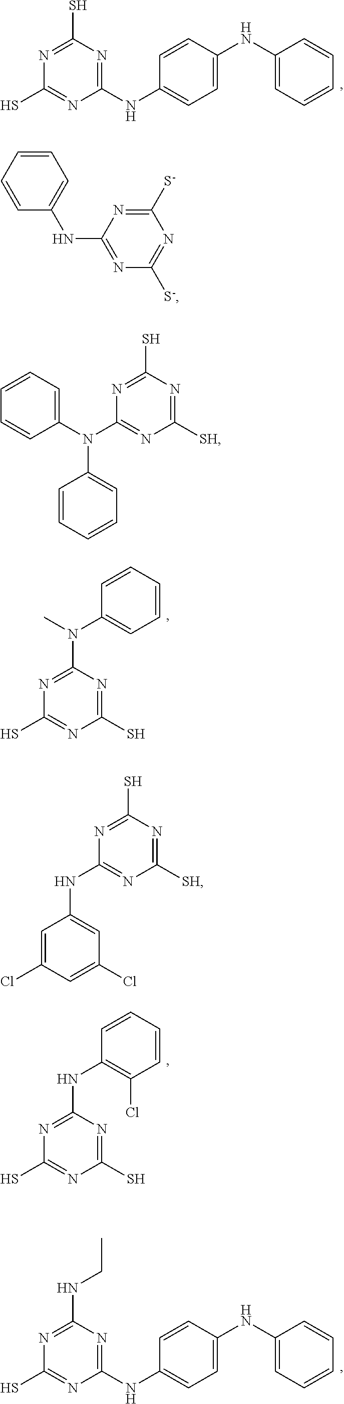

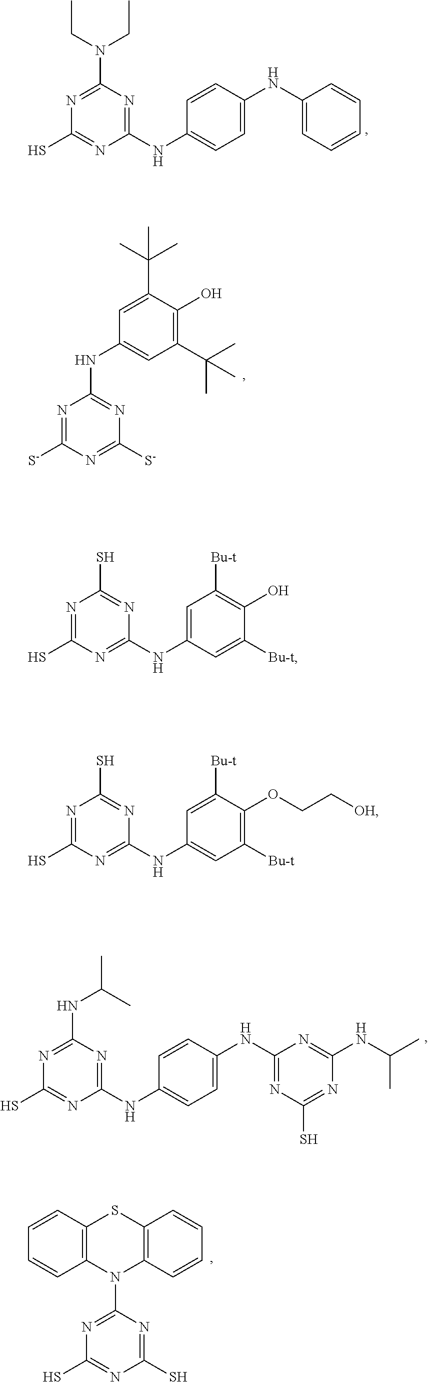

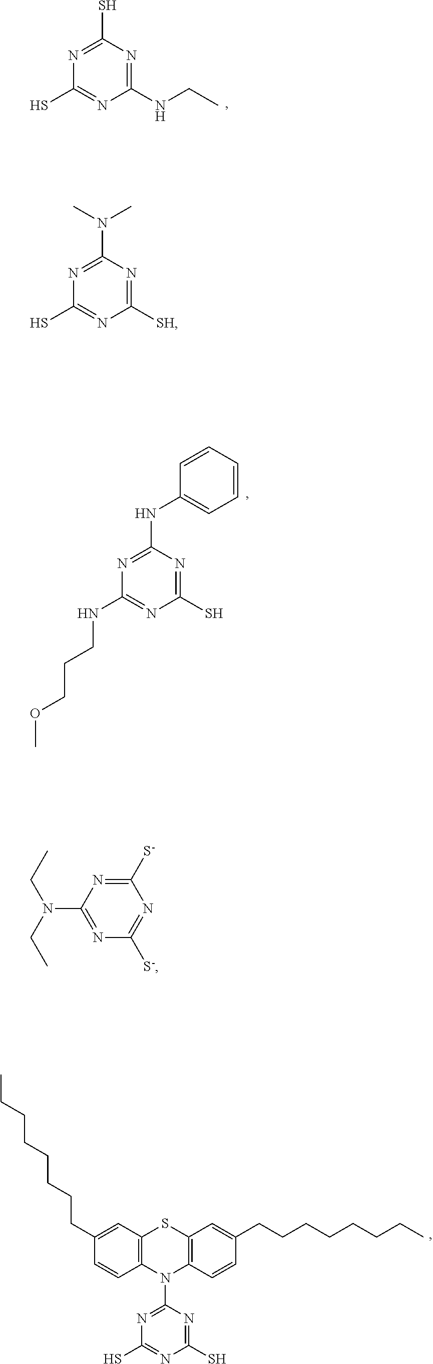

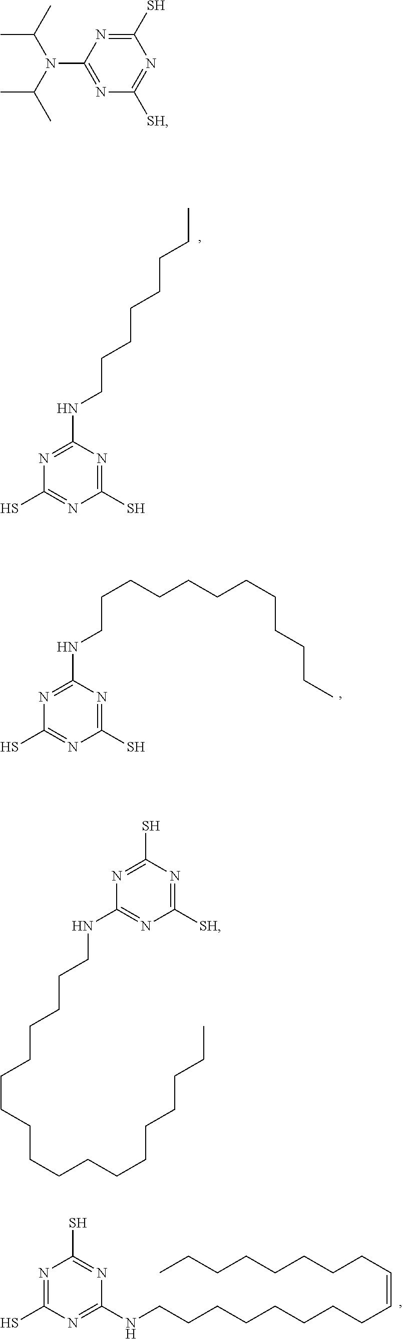 [SMILES: CC(C)N(c1nc(S)nc(S)n1)C(C)C ; CCCCCCCCNc1nc(S)nc(S)n1 ; CCCCCCCCCCCCNc1nc(S)nc(S)n1 ; CCCCCCCCCCCCCCCCCCNc1nc(S)nc(S)n1 ; CCCCCCCC/C=C\CCCCCCCCNc1nc(S)nc(S)n1]

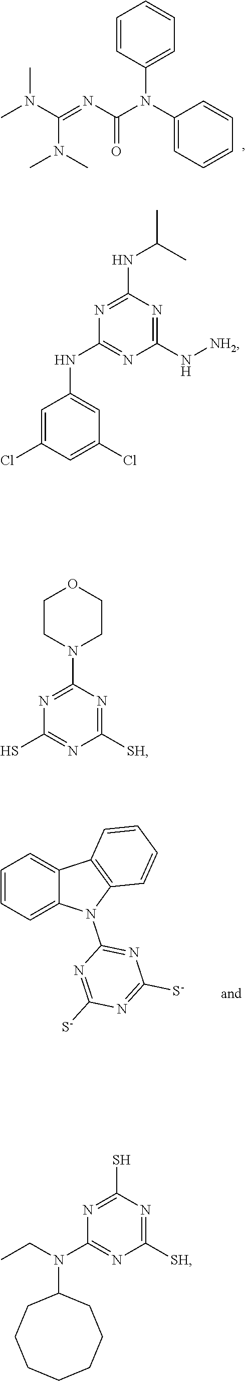 [SMILES: CN(C)C(=NC(=O)N(c1ccccc1)c1ccccc1)N(C)C ; CC(C)Nc1nc(NN)nc(Nc2cc(Cl)cc(Cl)c2)n1 ; Sc1nc(S)nc(N2CCOCC2)n1 ; [S-]c1nc([S-])nc(-n2c3ccccc3c3ccccc32)n1 ; CCN(c1nc(S)nc(S)n1)C1CCCCCCC1]

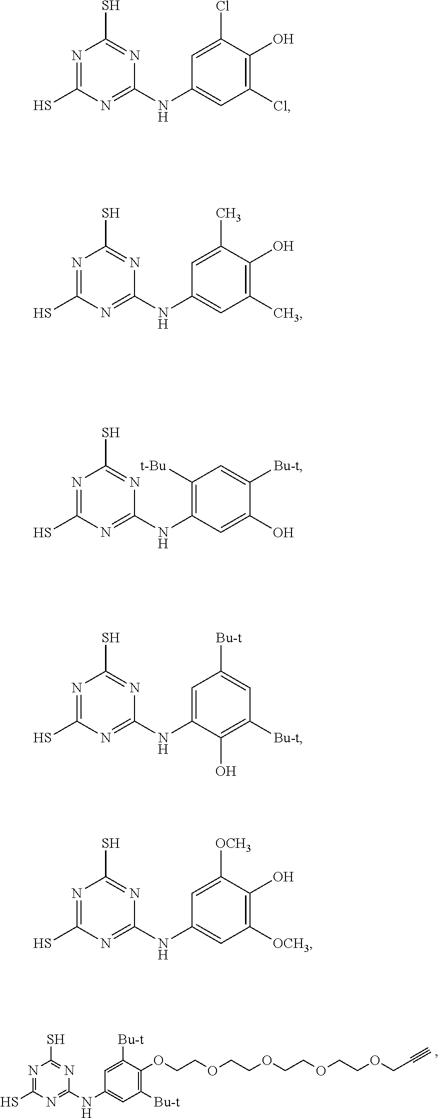

and to pharmaceutically acceptable salts individually thereof.

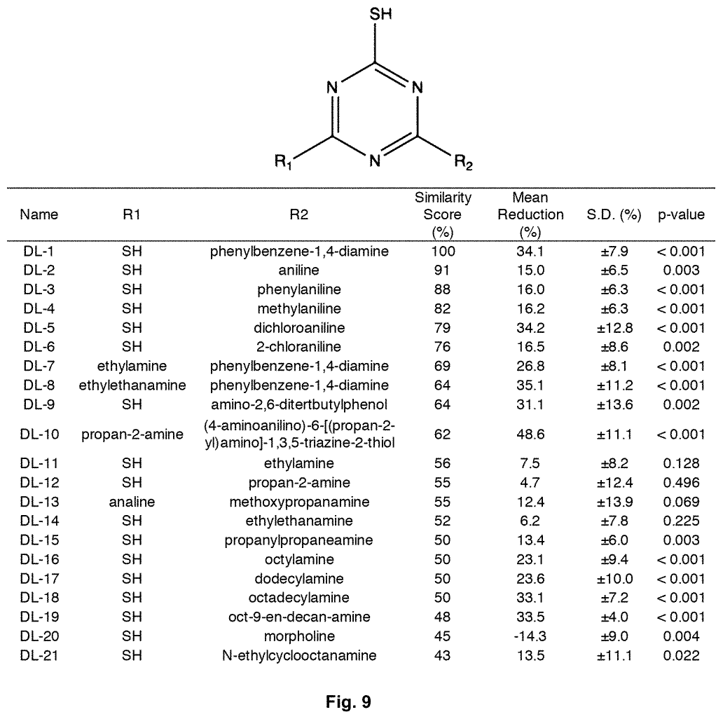

In one embodiment, the invention is directed to at least one of the compounds recited in FIG. 9 and to pharmaceutically acceptable salts individually thereof.

In another embodiment of the invention, and as shown in the Examples, representative compounds of the invention include:
N¹-(4,6-dichloro-1,3,5-triazin-2-yl)-N⁴-phenylbenzene-1,4-diamine;
S,S′-(6-((4-(phenylamino)phenyl)amino)-1,3,5-triazine-2,4-diyl) diethanethioate;
6-((4-(phenylamino)phenyl)amino)-1,3,5-triazine-2,4-dithiol;
S,S′-(6-((3-methoxypropyl)amino)-1,3,5-triazine-2,4-diyl)diethanethioate;
6-(phenylamino)-1,3,5-triazine-2,4-dithiol; or
N1-(4,6-dichloro-1,3,5-triazin-2-yl)-N4-phenylbenzene-1,4-diamine,
or a pharmaceutically acceptable salt thereof.

In another embodiment of the invention, and as shown in the Examples, representative compounds of the invention include:
6-{[4-(phenylamino)phenyl]amino}-1,3,5-triazine-2,4-dithiol;
[4-(phenylamino)-6-sulfanidyl-1,3,5-triazin-2-yl]sulfanide;
6-(diphenylamino)-1,3,5-triazine-2,4-dithiol;
6-[methyl(phenyl)amino]-1,3,5-triazine-2,4-dithiol;
6-[(3,5-dichlorophenyl)amino]-1,3,5-triazine-2,4-dithiol;
6-[(2-chlorophenyl)amino]-1,3,5-triazine-2,4-dithiol;
4-(ethylamino)-6-{[4-(phenylamino)phenyl]amino}-1,3,5-triazine-2-thiol;
4-(diethylamino)-6-{[4-(phenylamino)phenyl]amino}-1,3,5-triazine-2-thiol;
{4-[(3,5-di-tert-butyl-4-hydroxyphenyl)amino]-6-sulfanidyl-1,3,5-triazin-2-yl}sulfanide;
4-[(propan-2-yl)amino]-6-{[4-({4-[(propan-2-yl)amino]-6-sulfanyl-1,3,5-triazin-2-yl}amino)phenyl]amino}-1,3,5-triazine-2-thiol;
6-(ethylamino)-1,3,5-triazine-2,4-dithiol;
6-(dimethylamino)-1,3,5-triazine-2,4-dithiol;
4-[(3-methoxypropyl)amino]-6-(phenylamino)-1,3,5-triazine-2-thiol;
[4-(diethylamino)-6-sulfanidyl-1,3,5-triazin-2-yl]sulfanide;
6-[bis(propan-2-yl)amino]-1,3,5-triazine-2,4-dithiol;
6-(octylamino)-1,3,5-triazine-2,4-dithiol;
6-(dodecylamino)-1,3,5-triazine-2,4-dithiol;
6-(octadecylamino)-1,3,5-triazine-2,4-dithiol;
6-{[(9Z)-octadec-9-en-1-yl]amino}-1,3,5-triazine-2,4-dithiol;
6-(morpholin-4-yl)-1,3,5-triazine-2,4-dithiol;
6-[cyclooctyl(ethyl)amino]-1,3,5-triazine-2,4-dithiol;
6-(3,5-di-tert-butyl-4-(2-hydroxyethoxy)-phenylamino)-1,3,5-triazine-2,4-dithiol;
6-(3,5-dichloro-4-hydroxyphenylamino)-1,3,5-triazine-2,4-dithiol;
6-(3,5-dimethyl-4-hydroxyphenylamino)-1,3,5-triazine-2,4-dithiol;
6-(2,4-di-tert-butyl-5-hydroxyphenylamino)-1,3,5-triazine-2,4-dithiol;
6-(3,5-di-tert-butyl-2-hydroxyphenylamino)-1,3,5-triazine-2,4-dithiol;
6-(3,5-di-tert-butyl-4-(2-(2-(2-(2-(2-propyn-1-yloxy)ethoxy)ethyoxy)etoxy)ethoxy)-phenylamino)-1,3,5-triazine-2,4-dithiol; or
6-(3,5-di-tert-butyl-4-(2-(2-(2-(2-(2-propyn-1-yloxy)ethoxy)ethyoxy)etoxy)ethoxy)-phenylamino)-1,3,5-triazine-2,4-dithiol;
or a pharmaceutically acceptable salt thereof.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

A compound according to the invention is inherently intended to comprise all stereochemically isomeric forms thereof. The term “stereochemically isomeric forms” as used hereinbefore or hereinafter defines all the possible stereoisomeric forms which the compounds of formula (I) and their N-oxides, pharmaceutically acceptable salts or physiologically functional derivatives may possess. Unless otherwise mentioned or indicated, the chemical designation of compounds denotes the mixture of all possible stereochemically isomeric forms. In particular, stereogenic centers may have the R- or S-configuration; substituents on bivalent cyclic (partially) saturated radicals may have either the cis- or trans-configuration. Compounds encompassing double bonds can have an E (entgegen) or Z (zusammen)-stereochemistry at said double bond. The terms cis, trans, R, S, E and Z are well known to a person skilled in the art.

Stereochemically isomeric forms of the compounds of formula (I) are obviously intended to be embraced within the scope of this invention. Of special interest are those compounds of formula (I) which are stereochemically pure.

Following CAS-nomenclature conventions, when two stereogenic centers of known absolute configuration are present in a molecule, an R or S descriptor is assigned (based on Cahn-Ingold-Prelog sequence rule) to the lowest-numbered chiral center, the reference center. The configuration of the second stereogenic center is indicated using relative descriptors [R*,R*] or [R*,S*], where R* is always specified as the reference center and [R*,R*] indicates centers with the same chirality and [R*,S*] indicates centers of unlike chirality. For example, if the lowest-numbered chiral center in the molecule has an S configuration and the second center is R, the stereo descriptor would be specified as S—[R*,S*]. If “α” and “β” are used: the position of the highest priority substituent on the asymmetric carbon atom in the ring system having the lowest ring number, is arbitrarily always in the “α” position of the mean plane determined by the ring system. The position of the highest priority substituent on the other asymmetric carbon atom in the ring system relative to the position of the highest priority substituent on the reference atom is denominated “α”, if it is on the same side of the mean plane determined by the ring system, or “β”, if it is on the other side of the mean plane determined by the ring system.

When a specific stereoisomeric form is indicated, this means that said form is substantially free, i.e. associated with less than 50%, preferably less than 20%, more preferably less than 10%, even more preferably less than 5%, further preferably less than 2% and most preferably less than 1% of the other isomer(s). Thus, when a compound of formula (I) is for instance specified as (R,S), this means that the compound is substantially free of the (S,R) isomer.

The compounds of formula (I) may be synthesized in the form of mixtures, in particular racemic mixtures, of enantiomers which can be separated from one another following art-known resolution procedures. The racemic compounds of formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

The tautomeric forms of the compounds of formula (I) are meant to comprise those compounds of formula (I) wherein e.g. an enol group is converted into a keto group (keto-enol tautomerism). Tautomeric forms of the compounds of formula (I) or of intermediates of the present invention are intended to be embraced by the ambit of this invention.

The term “alkyl” as used herein denotes an unbranched or branched chain, saturated, monovalent hydrocarbon residue containing 1 to 20 carbon atoms. In one embodiment, the number of carbon atoms in the alkyl chain can be 2, 3, 4, 5, 6, 7, 8, 9, 10, 11, 12, 13, 14, 15, 16, 17, 18, 19 or 20 carbon atoms. In another embodiment, the number of carbon atoms in the alkyl chain can be from 5 to 16 and referred to as “(C₅-C₁₆)alkyl.” The term “lower alkyl” denotes a straight or branched chain hydrocarbon residue containing 1 to 6 carbon atoms. “C₁₋₂₀ alkyl” as used herein refers to an alkyl composed of 1 to 20 carbons. Examples of alkyl groups include, but are not limited to, lower alkyl groups include methyl, ethyl, propyl, i-propyl, n-butyl, i-butyl, t-butyl, pentyl, isopentyl, neopentyl, hexyl, heptyl, octyl, nonyl, decyl, undecyl, dodecyl, tridecycl and hexadecyl.

The term “alkenyl” as used herein denotes an unbranched or branched chain, saturated, monovalent hydrocarbon residue of from 2 to 24 carbon atoms with a structural formula containing at least one carbon-carbon double bond. In one embodiment, the number of carbon atoms in the alkenyl chain can be 2, 3, 4, 5, 6, 7, 8, 9, 10, 11, 12, 13, 14, 15, 16, 17, 18, 19 or 20 carbon atoms. In another embodiment, the number of carbon atoms in the alkenyl chain can be from 5 to 16 and referred to as “(C₅-C₁₆)alkenyl.”

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″-, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “halogen” as used herein means fluorine, chlorine, bromine or iodine. In one embodiment, halogen may be chlorine.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthalene, 1,2-dihydronaphthalene, indanyl, 1H-indenyl and the like.

The term “cycloalkyl” refers to a monovalent mono- or polycarbocyclic radical of three to ten, in one embodiment three to six, carbon atoms. This term is further exemplified by radicals such as cyclopropyl, cyclobutyl, cyclopentyl, cyclohexyl, cycloheptyl, norbornyl, adamantyl, indanyl and the like. In one embodiment, the “cycloalkyl” moieties can optionally be substituted with one, two, three or four substituents. Each substituent can independently be, alkyl, alkoxy, halogen, amino, hydroxyl or oxygen unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate.

The alkyl, lower alkyl, aryl, cycloalkyl and heterocycloalkyl groups may be substituted or unsubstituted. When substituted, there will generally be, for example, 1 to 4 substituents present. These substituents may optionally form a ring with the alkyl, lower alkyl or aryl group with which they are connected. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl, in another embodiment, for example, methoxy and ethoxy), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group.

The heteroaryl group described above may be substituted independently with one, two, or three substituents. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl, benzothiazoyl and carbolinyl).

A “patient” is a mammal, e.g., a human, mouse, rat, guinea pig, dog, cat, horse, cow, pig, or non-human primate, such as a monkey, chimpanzee, baboon or rhesus monkey, and the terms “patient” and “subject” are used interchangeably herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “optionally substituted,” as used in this disclosure, means a suitable substituent can replace a hydrogen bound to a carbon, nitrogen, or oxygen. When a substituent is oxo (i.e., ═O) then 2 hydrogens on the atom are replaced by a single O. In one embodiment, an alkyl or lower alkyl group can substituted with, for example, —N₃, —C≡CH, phenyl or OH. It will be understood by those skilled in the art, with respect to any group containing one or more substituents, that such groups are not intended to introduce any substitution or substitution patterns that are sterically impractical, synthetically non-feasible and/or inherently unstable. Furthermore, combinations of substituents and/or variables within any of the Formulae represented herein are permissible only if such combinations result in stable compounds or useful synthetic intermediates wherein stable implies a reasonable pharmacologically relevant half-life at physiological conditions.

Dosage and Administration:

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well-known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to a skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polylactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight, and most preferred 1.0 and about 15 mg/kg body weight per day. Thus, for administration to a 70 kg person, the dosage range in one embodiment would be about 70 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Compounds of the present invention can be prepared beginning with commercially available starting materials and utilizing general synthetic techniques and procedures known to those skilled in the art. Chemicals may be purchased from companies such as for example SigmaAldrich, Argonaut Technologies, VWR and Lancaster. Chromatography supplies and equipment may be purchased from such companies as for example AnaLogix, Inc., Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; and Waters Corporation, Milford, Mass. Biotage, ISCO and Analogix columns are pre-packed silica gel columns used in standard chromatography.

### EXAMPLES

The following examples further describe and demonstrate particular embodiments within the scope of the present invention. Techniques and formulations generally are found in Remington's Pharmaceutical Sciences (Mack Publishing Co., Easton, Pa.). The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

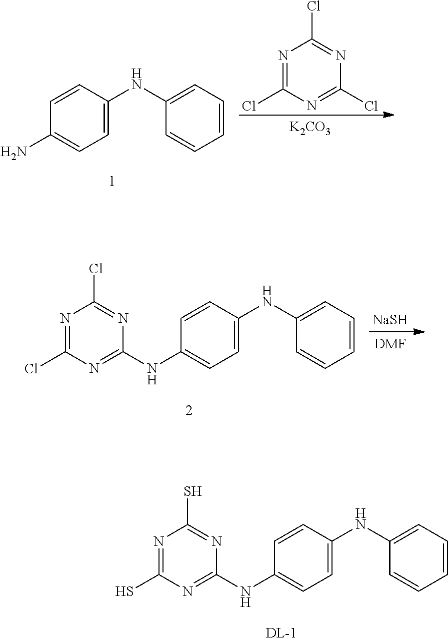

DL-1 was synthesized from commercially available N-phenyl-1,4-benzenediamine. The latter reacted with cyanuric chloride in the presence of anhydrous potassium carbonate at room temperature under nitrogen atmosphere, affording compound 2 in 61% yield. The compound 2 reacted with sodium hydrosulfide (NaSH) in dimethylformamide (DMF) at 90° C., producing crude DL-1. The product was recrystallized from DMF, affording DMF containing DL-1. The residual DMF was removed by dissolving the DMF containing DL-1 in dimethyl sulfoxide (DMSO) in small volume by heating, pouring the DMSO solution into water and collecting the white precipitate by filtration. After drying in the air, purified DL-1 was resulted in 88% yield. The compounds 2 and DL-1 were characterized by proton NMR spectroscopy and ESI TOF mass spectrometry with high accuracy.

### Example 2

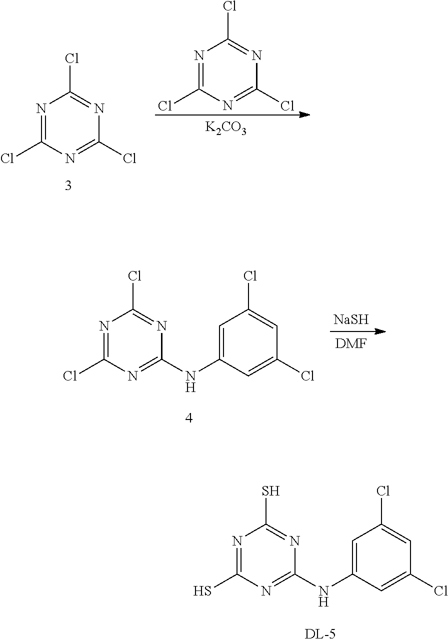

DL-5 was synthesized from commercially available 3,5-dichloroaniline, which reacted with cyanuric chloride in the presence of anhydrous potassium carbonate at room temperature under nitrogen atmosphere, affording compound 4 in 62% yield. Compound 4 reacted with NaSH in DMF, affording crude DL-5, which was purified by recrystallization from DMF, re-dissolving into DMSO and precipitating from water. This step resulted in DL-5 in 53% yield. The process is similar to that for production of DL-1. The compounds 4 and DL-5 were characterized by proton NMR spectroscopy and ESI TOF mass spectrometry with high accuracy.

### Example 3

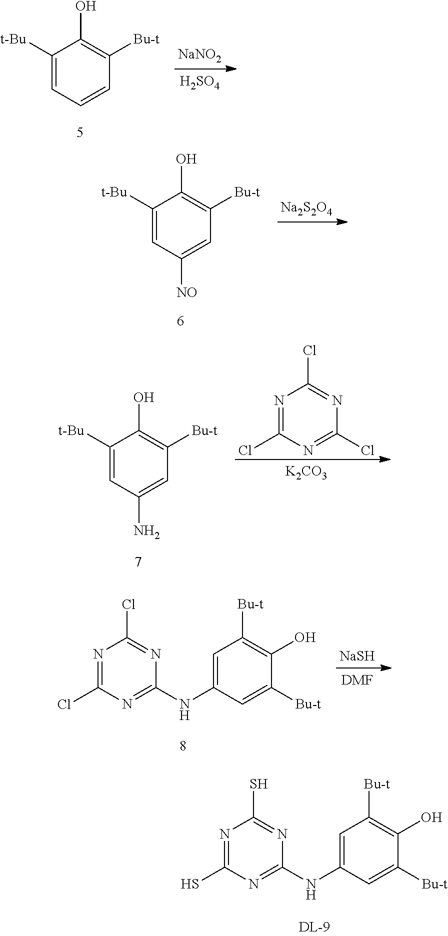

DL-9 was synthesized from the commercially available 2,6-di-tert-butylphenol, which was reacted with nitrous acid from the reaction of sodium nitrite with sulfuric acid at 0° C., affording crude compound 6. The compound 6 was purified by simple washing with dichloromethane during filtration. This step has 69% yield for compound 6.

The compound 6 was reduced to compound 7 by using sodium hydrosulfite in a solution of sodium hydroxide (5% NaOH) at 50-60° C., resulting in precipitate and compound 7. However, compound 7 was not stable in the air. It was produced just before use in the next step. To reduce the complication and oxidation, the precipitate was filtered and washed with water. The wet compound 7 was dissolved in dichloromethane and dried over anhydrous sodium sulfate. After the organic layer was concentrated in vacuum, the solvent free compound 7 was ready for next step without further purification.

The compound 7 then reacted with cyanuric chloride in the presence of anhydrous potassium carbonate at room temperature under nitrogen atmosphere, affording compound 8 in total 62% yield for the two steps. Compound 8 reacted with NaSH in DMF, affording crude DL-9, which was purified by recrystallization from DMF, re-dissolving into DMSO and precipitating from water. This step resulted in DL-9 in 49% yield. The process is also similar to that for production of DL-1. The compound 6 was characterized by proton NMR spectroscopy. The compounds 8 and DL-9 were characterized by proton NMR spectroscopy and ESI TOF mass spectrometry with high accuracy.

### Example 4

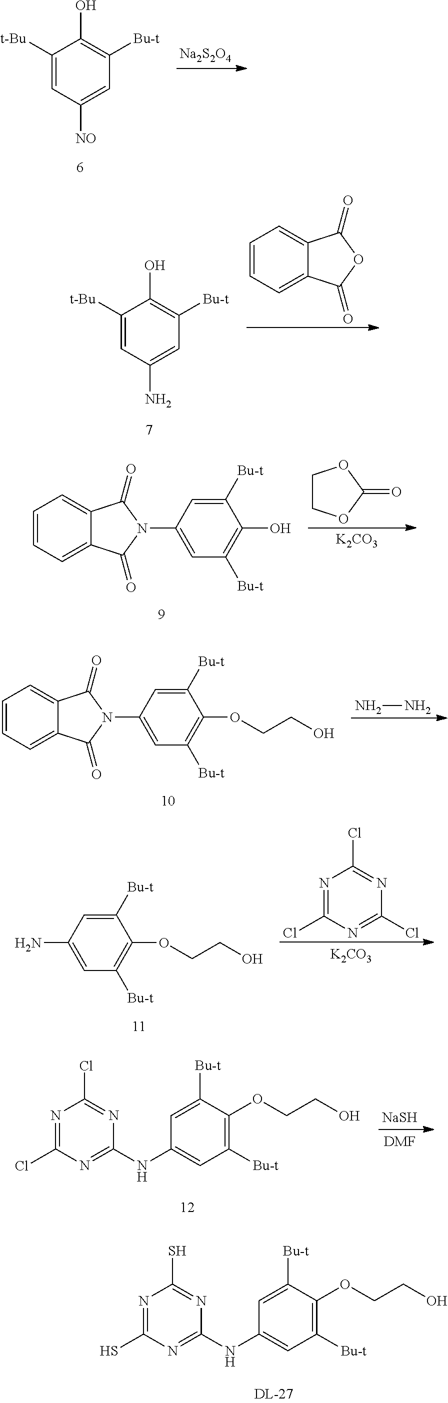

DL-27 was synthesized from compound 6, which was first reduced to compound 7 using sodium hydrosulfite. The compound 7 then reacted with phthalic anhydride in the presence of hydrochloric acid by reflux without delay to produce compound 9. This process is to protect the amino group of the compound 7, which is not stable. Unreacted phthalic anhydride was removed by treating with 5% sodium bicarbonate at 60° C. for 1 h in a solution of methanol and acetone. Compound 9 was purified by crystallization from ethyl acetate by evaporating of the solvent in vacuum and washing of the crystals with a mixed solvent of acetone with hexanes (⅓, volume/volume). This step resulted in compound 9 in 52% yield.

Compound 9 reacted with ethylene carbonate by refluxing in DMF, resulting in compound 10 in 17% yield. The deprotection of compound 10 was carried out using hydrazine at 65° C. for 2 h, affording compound 11. The compound 11 then reacted with cyanuric chloride in the presence of anhydrous potassium carbonate at room temperature under nitrogen atmosphere, affording compound 12 in total 47% yield for the two steps from compound 10. Compound 12 reacted with NaSH in DMF at 0° C. 1 h and 60° C. for 1 h, affording DL-27 in 77% yield. The compounds 9 and DL-27 were characterized by proton NMR spectroscopy and ESI TOF mass spectrometry with high accuracy. The compound 10 was characterized by proton NMR spectroscopy.

### Example 5

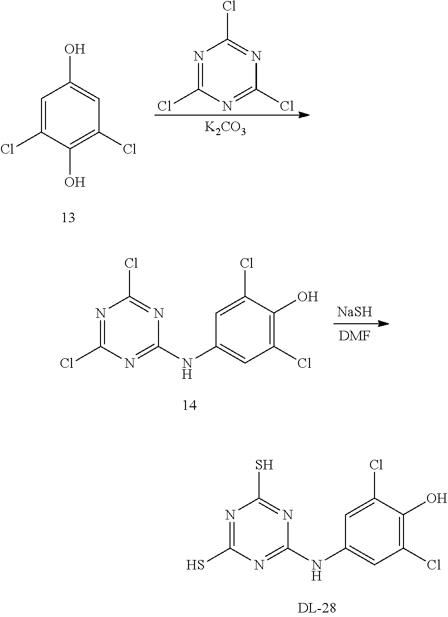

DL-28 was synthesized from commercially available 4-amino-2,6-dichlorophenol, which was reacted with cyanuric chloride in the presence of anhydrous potassium carbonate at room temperature under nitrogen atmosphere, affording compound 14 in 60% yield. Compound 14 reacted with NaSH in DMF at 0° C. for 1 h then 60° C. for 1 h, affording DL-28 in 94% yield. The compounds 14 and DL-28 were characterized by proton NMR spectroscopy and ESI TOF mass spectrometry with high accuracy.

### Example 6

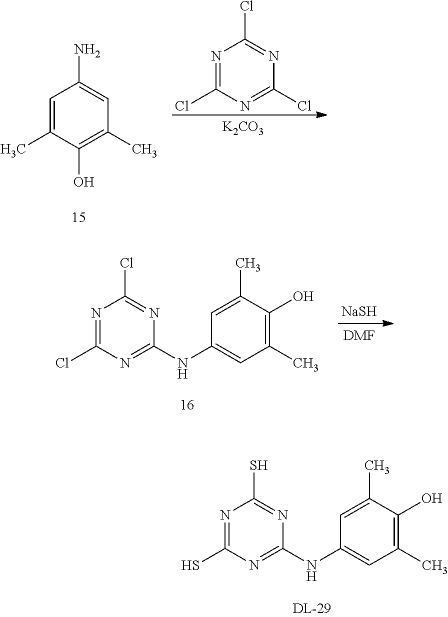

DL-29 was synthesized from commercially available 4-amino-2,6-dimethylphenol, which was reacted with cyanuric chloride in the presence of anhydrous potassium carbonate at room temperature under nitrogen atmosphere, affording compound 16 in 78% yield. Compound 16 reacted with NaSH in DMF at 0° C. for 1 h then 60° C. for 1 h, affording DL-29 in 78% yield. The compounds 16 and DL-29 were characterized by proton NMR spectroscopy and ESI TOF mass spectrometry with high accuracy.

### Example 7

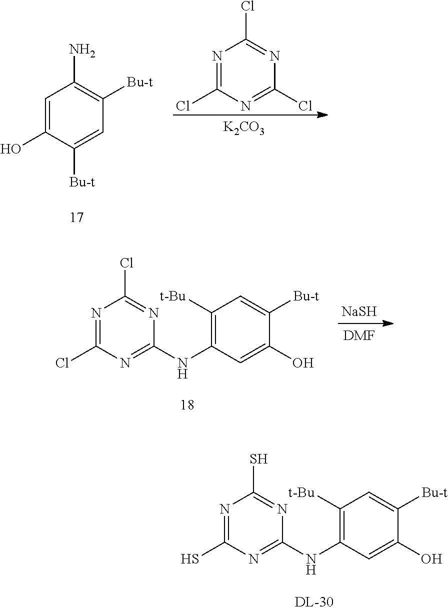

DL-30 was synthesized from commercially available 3-amino-4,6-di-tert-butylphenol, which was reacted with cyanuric chloride in the presence of anhydrous potassium carbonate at room temperature under nitrogen atmosphere, affording compound 18 in 78% yield. Compound 18 reacted with NaSH in DMF at 0° C. for 1 h then 60° C. for 1 h, affording DL-30 in 98% yield. The compounds 18 and DL-30 were characterized by proton NMR spectroscopy and ESI TOF mass spectrometry with high accuracy.

### Example 8

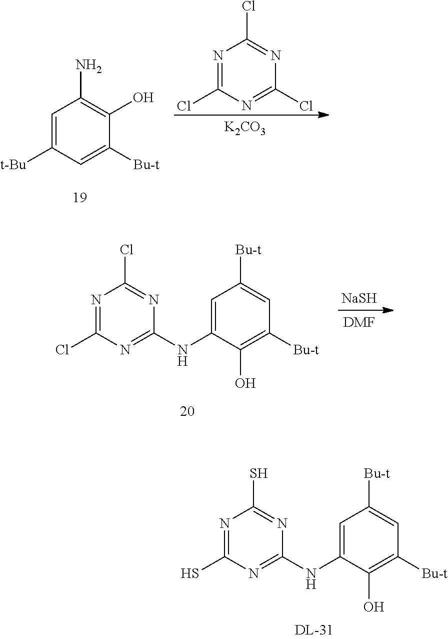

DL-31 was synthesized from commercially available 2-amino-4,6-di-tert-butylphenol, which was reacted with cyanuric chloride in the presence of anhydrous potassium carbonate at room temperature under nitrogen atmosphere, affording compound 20 in 100% yield. Compound 20 reacted with NaSH in DMF at 0° C. for 1 h then 60° C. for 1 h, affording DL-31 in 98% yield. The compounds 20 and DL-31 were characterized by proton NMR spectroscopy and ESI TOF mass spectrometry with high accuracy.

### Example 9

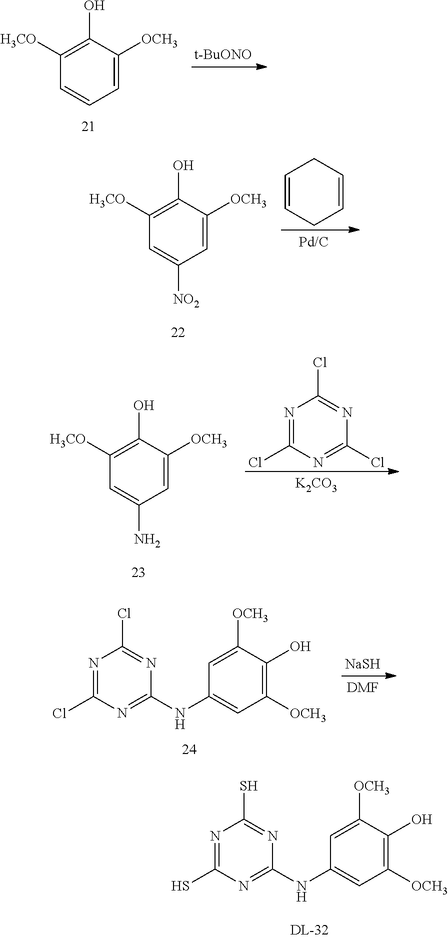

DL-32 was synthesized from commercially available 2,6-dimethoxyphenol, which was reacted with tert-butyl nitrite in tetrahydrofuran (THF) at room temperature, affording compound 22 in 14% yield. Compound 22 was reduced to compound 23 with 1,4-cyclohexadiene in the presence of palladium on carbon at room temperature. The compound 23 then reacted with cyanuric chloride in the presence of anhydrous potassium carbonate at room temperature under nitrogen atmosphere, affording compound 24 in 43% yield for two steps from compound 22. Compound 24 reacted with NaSH in DMF at 0° C. for 1 h then 60° C. for Ih, affording DL-32 in 93% yield. The compounds 24 and DL-32 were characterized by proton NMR spectroscopy and ESI TOF mass spectrometry with high accuracy.

### Example 10

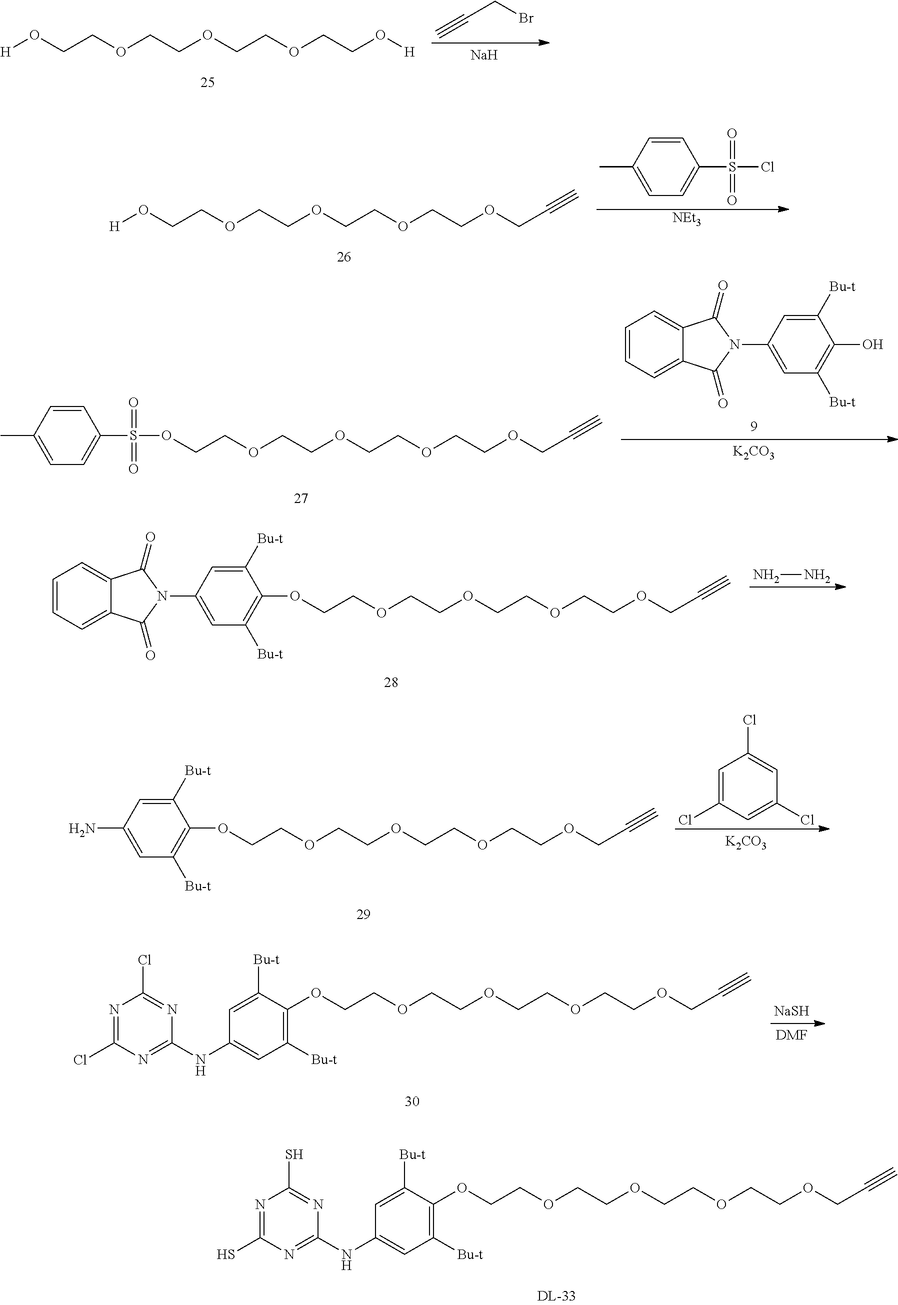

DL-33 was synthesized starting from commercially available tetraethylene glycol, which was reacted with propargyl bromide in the presence of sodium hydride in THF at −10° C., affording compound 26 in 42% yield. The compound 26 then reacted with tosyl chloride in the presence of triethylamine, resulting in compound 27. The compound 27 reacted with compound 9 in the presence of anhydrous potassium carbonate to make compound 28. When 2-butanone was used as a solvent and 2.5 equivalent of potassium carbonate used as base, the yield was 18%. The reaction was improved by using acetonitrile as a solvent and 5 equivalent of base potassium carbonate, resulting in compound 28 in 33% yield.

The compound 28 then reacted with hydrazine in the presence of sodium hydroxide, affording compound 29 (94% yield), which reacted with cyanuric chloride in the presence of anhydrous potassium carbonate at room temperature under nitrogen atmosphere, affording compound 30 in 98% yield. Compound 30 reacted with NaSH in DMF at 0° C. for 1 h then 60° C. for 1 h, affording DL-33 in 86% yield. The compounds 28, 30 and DL-33 were characterized by proton NMR spectroscopy and ESI TOF mass spectrometry with high accuracy.

### Example 11

The molar extinction co-efficients of exemplary cholesterol lowering compounds were measured in 1 x PBS buffer. The compounds were first dissolved in dimethyl sulfoxide. Solutions with a 0.5 mg/ml concentration were then prepared in DMSO. These solutions were diluted further with 1 x PBS by 100-fold, resulting in solutions with a 5 ug/ml concentration. The resulted diluted solutions were then measured and scanned using a UV-vis spectrophotometer for ultra-violet absorbances between 240 nm to 400 nm, using a UV 96-well microplate. The maximum UV absorbances and corresponding wavelengths were recorded (Table 1). The absorbance values were subtracted by that of a blank, 1 x PBS buffer containing DM. The volume of the solutions for UV measurements was 300 ul and the depth of the solutions was 0.7 cm. The molar extinction co-efficients were then calculated based on formula

A=ϵCL. (1)

whereas A, UV absorbance; c, extinction co-efficient; C, concentration and L, length (0.7 cm).

TABLE 1
The wavelengths at the maximum UV absorbances and extinction
coefficients of the cholesterol lowering compounds, and their solubilities
in water and 1 × PBS buffer at 25° C.

| | | | | | Solubility |
|---|---|---|---|---|---|
| | | | Extinction | Solubility | in 1.0 ml |
| | Molecular | UVmax | coefficient | in 1.0 ml | PBS buffer |
| Compounds | weight | (nm) | (mM⁻¹CM⁻¹) | water (μg) | (μg) |
| DL-1 | 327 | 274 | 55.9 | 0.37 | 25 |
| DL-5 | 305 | 285 | 61.9 | 0.27 | 61 |
| DL-9 | 364 | 275 | 51.0 | 0.40 | 91 |
| DL-27 | 408 | 277 | 50.4 | 1.78 | 257 |
| DL-28 | 319 | 274 | 47.4 | 1.25 | 348 |
| DL-29 | 280 | 274 | 53.7 | 2.22 | 686 |
| DL-30 | 364 | 270 | 46.2 | 1.35 | 459 |
| DL-31 | 364 | 275 | 44.2 | 1.63 | 477 |
| DL-32 | 312 | 275 | 53.3 | 2.01 | 786 |
| DL-33 | 579 | 277 | 47.0 | 1.18 | 186 |

The solubilities of the exemplary cholesterol lowering compounds were measured in water and 1 x PBS buffer. The compounds were first stirred with water and 1 x PBS buffer individually at room temperature for 1 h. The suspensions were then centrifuged for 2 min. The top clear solutions were filtered through filter paper, resulting in mother liquids, which were centrifuged again and diluted with 1 x PBS buffer for 10-fold for the solubilities in water and 100-fold the solubilities in 1 x PBS buffer. The resulted solutions were measured using a UV-vis spectrophotometer for UV absorbances with the maximum absorbances. The values were subtracted by that of a blank, 1 x PBS buffer. The corrected absorbances were then used to calculate the corresponding concentrations using the formula (1) and extinction coefficients (Table 1) shown above. The individual concentrations were then used to calculate the solubilities in 1.0 mL of water and 1 x PBS buffer. The results were listed in Table 1 as well.

As shown in Table 1, indeed, DL-9 has the best solubility among the first three compounds (DL-1, 5 and 9) in both water or 1 x PBS buffer. Surprisingly, DL-27, DL-28, DL-29, DL-30, DL-31, DL-32, and DL-33 have better solubilities than that for DL-9. For example, the solubility of DL-31 is about 4-fold of that of DL-9 in water, and 5-fold in 1 x PBS buffer.

### Experimental Details for Examples 1-11

General: All chemicals were purchased from various commercial sources and used without further purification. Flash chromatography was performed with silica gel (70-230 mesh from Sorbent Technologies) and monitored by thin layer chromatography (TLC) with silica gel plates (Merck, Kieselgel 60 F₂₅₄). The ¹H spectra were recorded on a 400 MHz Bruker instrument. The spectra were recorded in hexadeuterioacetone (CD₃COCD₃) or hexadeuteriodimethylsulfoxide (DMSO-d₆). Chemical shifts of protons are given in parts per million with the solvent as internal standard. ESI-TOF high accuracy spectra were recorded on an Agilent 6230 TOF LC/MS system.

Synthesis of compound 2: To a solution of cyanuric chloride (2.31 g, 12.5 mmol) in tetrahydrofuran (THF, 12 mL), was added anhydrous potassium carbonate (3.46 g, 25 mmol) under nitrogen, while stirring with a magnetic stirrer. To this suspension, N-phenyl-1,4-benzenediamine (2.31 g, 2.5 mmol) solution in tetrahydrofuran (13 ml) was then added dropwise through an addition funnel within 20 min. The resulted mixture was stirred overnight at room temperature, and then filtered through Celite. The mother liquid was concentrated under vacuum, and the residue was submitted to purification with column chromatography using silica gel and solvent system from hexanes to 70/30 (v/v, volume/volume) dichloromethane/hexanes, affording compound 2 as an off-gray solid (2.55 g, 61%). ¹H NMR (DMSO-d⁶, ppm) δ 6.83 (t, 1H, J=8 Hz, Ar—H); 7.06-7.11 (m, 4H, Ar—H); 7.21-7.26 (m, 2H, Ar—H); 7.42 (d, 2H, J=8 Hz, Ar—H), 8.22 (s, 1H, N—H); 10.97 (s, 1H, N—H). ESI-TOF high accurate mass (M+H⁺) for C₁₅H₁₂N₅Cl₂, calculated, 332.046; found, 332.047.

Synthesis of DL-1: A solution of compound 2 (2.83 g, 8.5 mmol) in dimethylformamide (DMF, 10 mL) was prepared first under nitrogen while stirring. This solution was then cooled to 0° C. on ice-water bath and water (1.36 ml) was added. To this solution, a solution of sodium hydrosulfide (NaSH, 2.125 g, 25.5 mmol) in water (5.0 mL) was added within 2 h. After the completion of the addition, the resulted mixture was stirred at 0° C. for 1 h, and 90° C. for 1 h. After cooling at room temperature, the mixture was poured on to water (70 mL) and acidified with 3 N HCl to pH 3. The resulting precipitate was filtered, washed with water and recrystallized from DMF (25 mL) by heating, affording DMF containing solid (3.62 g). This solid was dissolved in DMSO (9 mL) by heating. The resulting solution was added onto water (400 mL) while stirring. The resulting precipitate was filtered, washed with water and dried in the air, affording DMF free DL-1 as a light yellow solid (2.44 g, 88%). ¹H NMR (DMSO-d⁶, ppm) δ 6.83 (t, 1H, J=8 Hz, Ar—H); 7.06-7.11 (m, 4H, Ar—H); 7.21-7.26 (m, 2H, Ar—H); 7.41 (d, 2H, J=8 Hz, Ar—H), 8.24 (s, 1H, N—H); 9.08 (s, 1H, N—H); 12.03 (s, 1H, S—H), 13.06 (s, 1H, S—H). ESI-TOF high accurate mass (M+H⁺) for C₁₅H₁₄N₅S₂(M+H⁺), calculated: 328.069; found: 328.069.

Synthesis of compound 4: To a solution of cyanuric chloride (1.84 g, 10.0 mmol) in THE (10 mL), was added anhydrous potassium carbonate (2.76 g, 20 mmol) under nitrogen, while stirring with a magnetic stirrer. To this suspension, 3,5-dichloroaniline (1.62 g, 10.0 mmol) solution in tetrahydrofuran (10 ml) was then added dropwise through an addition funnel within 20 min. The resulted mixture was stirred overnight at room temperature, and then filtered through Celite. The mother liquid was concentrated under vacuum, and the residue was submitted to purification with column chromatography using silica gel and solvent system from hexanes to 50/50 (v/v, volume/volume) dichloromethane/hexanes, affording compound 4 as a white solid (1.93 g, 62%).¹H NMR (DMSO-d⁶, ppm) δ 7.42 (t, 1H, J=4 Hz, Ar—H); 7.72 (d, 2H, J=4 Hz, Ar—H), 11.43 (s, 1H, N—H). ESI-TOF high accurate mass (M+H⁺) for C₉H₅N₄Cl₄, calculated, 308.926; found, 308.926.

Synthesis of DL-5: A solution of compound 4 (1.55 g, 5.0 mmol) in DMF (14 mL) was prepared first under nitrogen while stirring. This solution was then cooled to 0° C. on ice-water bath and water (0.8 mL) was added. To this solution, a solution of sodium hydrosulfide (1.25 g, 15.0 mmol) in water (2.5 mL) was added within 2 h. After the completion of the addition, the resulted mixture was stirred at 0° C. for 1 h, and 90° C. for 1 h. After cooling, the mixture was poured on to water (70 mL) and acidified with 3 N HCl to pH 3. The resulting precipitate was filtered, washed with water and recrystallized from DMF (20 mL) by heating, affording DMF containing solid (1.38 g). This solid was dissolved in DMSO (16 mL) by heating. The resulting solution was added onto water (320 mL) while stirring. The resulting precipitate was filtered, washed with water and dried in the air, affording DMF free DL-5 as a white solid (0.86 g, 53%). ¹H NMR (DMSO-d⁶, ppm) δ 7.41 (t, 1H, J=4 Hz, Ar—H); 7.69 (s, 2H, Ar—H), 9.43 (s, 1H, N—H); 13.21 (s, 1H, S—H). ESI-TOF high accurate mass (M−H⁺) for C₉H₅N₄Cl₂S₂, calculated: 302.933; found: 302.926.

Synthesis of compound 6: To a solution of 2,6-di-tert-butylphenol (46.7 g, 227 mmol) in ethanol (200 mL), was added sulfuric acid (98%, 6.65 mL) and then a solution of sodium nitrite (16.15 g, 234 mmol) in water (50 mL) dropwise through an addition funnel at 0° C. using a salt-ice water bath. During the reaction and when the reaction mixture was difficult to stir by a magnetic stirrer, manual shaking was applied. After the completion of the addition, the reaction mixture was sat for 75 min below 10° C., filtered, and washed with water and then dichloromethane, affording pure compound 6 as a white solid (36.6 g, 69%) based on thin layer chromatography. ¹H NMR (DMSO-d⁶, ppm) δ 1.25 (s, 18H, C—H); 6.99 (d, 1H, J=4 Hz, Ar—H); 7.49 (d, 1H, J=4 Hz, Ar—H); 13.24 (s, 1H, O—H).

Synthesis of compounds 7 and 8: Compound 6 (2.35 g, 10.0 mmol) was dissolved in aqueous solution of sodium hydroxide (5%, 25 mL). The solution was heated to 50-60° C. To the hot solution, was added sodium hydrosulfite (4.5 g) by one portion. After the reaction mixture was maintained at this temperature for 1 h, 20 ml of water was added. The resulted mixture was then cooled to room temperature. The reaction mixture was filtered using a filter funnel and washed with water. The solid on the filter funnel was dissolved into dichloromethane. The organic layer was separated from aqueous layer using a separation funnel and dried over anhydrous sodium sulfate. The organic layer was then concentrated in vacuum, affording compound 7, which is preserved in nitrogen atmosphere and used in next step without further purification.

To a solution of cyanuric chloride (1.84 g, 10.0 mmol) in tetrahydrofuran (10 mL), was added anhydrous potassium carbonate (2.76 g, 20 mmol) under nitrogen, while stirring with a magnetic stirrer. To this suspension, a solution of compound 7 in tetrahydrofuran (10 ml) was then added dropwise through an addition funnel within 20 min. The resulted mixture was stirred overnight at room temperature, and then filtered through Celite. The mother liquid was concentrated under vacuum, and the residue was submitted to purification with column chromatography using silica gel and solvent system from hexanes to 40/60 (v/v, volume/volume) dichloromethane/hexanes, affording compound 8 as a white solid (2.42 g, 66% for two steps).¹H NMR (DMSO-d⁶, ppm) δ 1.38 (s, 18H, C—H); 7.00 (s, 1H, N—H); 7.42 (s, 2H, Ar—H); 10.82 (s, 1H, O—H). ESI-TOF high accurate mass (M+H⁺) for C₁₇H₂₃N₄OCl₂, calculated, 369.124; found, 369.126.

Synthesis of DL-9: A solution of compound 8 (1.845 g, 5.0 mmol) in dimethylformamide (6 mL) was prepared first under nitrogen while stirring. This solution was then cooled to 0° C. on ice-water bath and water (0.8 mL) was added. To this solution, a solution of sodium hydrosulfide (1.25 g, 15.0 mmol) in water (3.0 mL) was added within 2 h. After the completion of the addition, the resulted mixture was stirred at 0° C. for 1 h, and 90° C. for 1 h. After cooling, the mixture was poured on to water (40 mL) and acidified with 3 N HCl to pH 3. The resulting precipitate was filtered, washed with water and recrystallized from DMF (12 mL) by heating, affording DMF containing solid (1.23 g). This solid was dissolved in DMSO (4.5 mL) by heating. The resulting solution was added onto water (200 mL) while stirring. The resulting precipitate was filtered, washed with water and dried in the air, affording DMF free DL-9 as a white solid (0.90 g, 49%). ¹H NMR (DMSO-d⁶, ppm) δ 1.38 (s, 18H, C—H); 7.04 (s, 1H, N—H); 7.30 (s, 2H, Ar—H); 8.99 (s, 1H, O—H); 12.03 (s, 1H, S—H); 13.02 (s, 1H, S—H). ESI-TOF high accurate mass (M+H⁺) for C₁₇H₂₅N₄OS₂ (M+H⁺), calculated: 365.146; found: 365.147.

Synthesis of compounds 7 and 9: Compound 6 (27.6 g, 117 mmol) was dissolved in aqueous solution of sodium hydroxide (5%, 300 mL). The solution was heated to 50-60° C. To the hot solution, was added sodium hydrosulfite (52.85 g) by one portion. When two thirds of sodium hydrosulfite had been added, the mixture was difficult to stir with a magnetic stirrer. Manual shaking was then applied. After the reaction mixture was maintained between 50 and 75° C. for 1 h, 250 ml of water was added. The resulted mixture was then cooled to room temperature. The reaction mixture was filtered using a filter funnel and washed with water. The solid on the filter funnel was dissolved with dichloromethane. The organic layer was separated from aqueous layer using a separation funnel and dried over anhydrous sodium sulfate. The organic layer was then concentrated in vacuum, affording compound 7, which is preserved in nitrogen atmosphere and used in next step without further purification.

To a solution of compound 7 in tetrahydrofuran (THF, 300 mL), was added phthalic anhydride (17.2 g, 117 mmol) under nitrogen. After the resulted mixture was refluxed on an oil bath for 24 h, concentrated hydrochloric acid (36.5%, 1.5 mL) was added. The reaction mixture was further refluxed for 17 h. The mixture was then cooled to room temperature and concentrated in vacuum. The residue was dissolved in a mixed solvent of acetone (200 mL) with methanol (200 mL). To this mixture, sodium bicarbonate (5%, 130 mL) was added. The resulted mixture was stirred at 60° C. for 1 h to remove residual phthalic anhydride. After the reaction mixture was cooled to room temperature, it was concentrated in vacuum and extracted with ethyl acetate (3×150 mL). The organic layer was dried over anhydrous magnesium sulfate and concentrated in vacuum to a moment, when significant crystallization took place. The crystals were separated by filtration and washed with a mixed solvent of acetone with hexanes (⅓, v/v). The mother liquid was further concentrated, and crystals were filtered and washed with the mixed solvent. The process was repeated for one more time, affording compound 9 as a white solid (total 21.5 g, 52%).¹H NMR (acetone-d⁶, ppm) δ 1.48 (s, 18H, C—H); 6.34 (s, 1H, O—H); 7.29 (s, 2H, C—H); 7.89-7.96 (m, 4H, Ar—H). ESI-TOF high accurate mass (M+H⁺) for C₂₂H₂₆NO₃, calculated, 352.191; found, 352.191.

Synthesis of compound 10: To a mixture of compound 9 (0.64 g, 1.82 mmol), ethylene carbonate (0.32 g, 3.64 mmol) and DMF (2.5 mL), was added potassium bicarbonate (0.404 g, 2.92 mmol).

The resulted suspension was refluxed under nitrogen atmosphere for 2 h. After the mixture was cooled to room temperature, water (20 mL), acetone (20 mL) and sodium hydroxide (5%, 20 mL) were then added. This mixture was stirred for 5 minutes at room temperature, concentrated in vacuum and extracted with ethyl acetate (2×30 mL). The organic layer was dried over magnesium sulfate and then concentrated in vacuum. The residue was submitted to purification with column chromatography using silica gel and solvent system from hexanes to 20/80 (v/v) acetone/hexanes, affording compound 10 as a white solid (0.12 g, 17%). ¹H NMR (acetone-d⁶, ppm) δ 1.48 (s, 18H, C—H); 3.96-4.02 (m, 4H, C—H); 7.43 (s, 2H, C—H); 7.91-7.97 (m, 4H, Ar—H).

Synthesis of compounds 11 and 12: To a solution of compound 10 (0.24 g, 0.61 mmol) in methanol (15 mL) was added hydrazine (65%, 1.19 g). The resulted mixture was heated at 65° C. for 2 h. To the mixture, anhydrous ethanol (40 mL) was added. The resulted mixture was then concentrated in vacuum to almost dry. To the residue, more anhydrous ethanol (40 mL) was added. The resulted suspension was concentrated in vacuum again to almost dry. To the residue, dichloromethane (50 mL) was added. The suspension was filtered and washed with dichloromethane. The mother liquid was collected, dried over magnesium sulfate and concentrated in vacuum, affording compound 11, which is being used in next step without further purification.

To a solution of cyanuric chloride (0.132 g, 0.71 mmol) in tetrahydrofuran (5 mL), was added anhydrous potassium carbonate (0.195 g, 1.42 mmol) under nitrogen, while stirring with a magnetic stirrer. To this suspension, a solution of compound 11 in tetrahydrofuran (10 ml) was then added dropwise through an addition funnel within 20 min. The resulted mixture was stirred overnight at room temperature, and then filtered through Celite. The mother liquid was concentrated under vacuum, and the residue was submitted to purification with column chromatography using silica gel and solvent system from hexanes to 20/80 (v/v) ethyl acetate/hexanes, affording compound 12 as a white solid (0.119 g, 47% for two steps).

Synthesis of DL-27: A solution of compound 12 (0.119 g, 0.288 mmol) in dimethylformamide (3.0 mL) was prepared first under nitrogen while stirring. This solution was then cooled to 0° C. on ice-water bath and water (0.3 ml) was added. To this solution, a solution of sodium hydrosulfide (0.26 g) in water (0.6 mL) was added within 2.5 min. After the completion of the addition, the resulted mixture was stirred at 0° C. for 1 h, and 60° C. for 1 h. After cooling, the mixture was poured on to water (30 mL) and acidified with 3 N HCl to pH 3. The resulting precipitate was filtered, washed with water and dried in the air, affording compound DL-27 as a white solid (90.7 mg, 77%). ¹H NMR (DMSO-d⁶, ppm) δ 1.39 (s, 18H, C—H); 3.73-3.79 (m, 4H, C—H); 7.46 (s, 2 H, Ar—H); 7.46 (s, 1H, N—H); 9.12 (s, 1H, N—H); 13.10 (s, 1H, S—H). ESI-TOF high accurate mass (M+H⁺) for C₁₉H₂₉N₄O₂S₂(M+H⁺), calculated: 409.173; found: 409.174.

Synthesis of compound 14: To a solution of cyanuric chloride (0.92 g, 5.0 mmol) in tetrahydrofuran (10 mL), was added anhydrous potassium carbonate (1.38 g, 10 mmol) under nitrogen, while stirring with a magnetic stirrer. To this suspension, 4-amino-2,6-dichlorophenol (13, 0.89 g, 5.0 mmol) solution in tetrahydrofuran (10 ml) was then added dropwise through an addition funnel within 15 min. The resulted mixture was stirred overnight at room temperature, and then filtered through Celite. The mother liquid was concentrated under vacuum, and the residue was submitted to purification with column chromatography using silica gel and solvent system from hexanes to 12.5/87.5 (v/v) ethyl acetate/hexanes, affording compound 14 as a white solid (0.977 g, 60%). ¹H NMR (acetone-d⁶, ppm) δ 7.76 (s, 2H, Ar—H); 8.91 (s, 1H, N—H); 9.88 (s, 1H, O—H). ESI-TOF high accurate mass (M−H⁺) for C₉H₃N₄OCl₄, calculated, 322.907; found, 322.907.

Synthesis of DL-28: A solution of compound 14 (0.855 g, 2.62 mmol) in dimethylformamide (10 mL) was prepared first under nitrogen while stirring. This solution was then cooled to 0° C. on ice-water bath and water (0.6 mL) was added. To this solution, a solution of sodium hydrosulfide (0.66 g, 67%, 7.86 mmol) in water (1.5 mL) was added in two portions. After the completion of the addition, the resulted mixture was stirred at 0° C. for 1 h, and 60° C. for 1 h. After cooling, the mixture was poured on to water (100 mL) and acidified with 3 N HCl to pH 3. The resulting precipitate was filtered, washed with water and dried in the air, affording DL-28 as a white solid (0.796 g, 94%). ¹H NMR (DMSO-d⁶, ppm) δ 7.76 (s, 2H, Ar—H); 9.18 (s, 1H, N—H); 10.20 (s, 1H, O—H); 13.13 (s, 1H, S—H). ESI-TOF high accurate mass (M−H⁺) for C₉H₇N₄OCl₂S₂, calculated: 320.943; found: 320.944.

Synthesis of compound 16: To a solution of cyanuric chloride (0.92 g, 5.0 mmol) in tetrahydrofuran (10 mL), was added anhydrous potassium carbonate (1.38 g, 10 mmol) under nitrogen, while stirring with a magnetic stirrer. To this suspension, 4-amino-2,6-dimethylphenol (15, 0.685 g, 5.0 mmol) solution in tetrahydrofuran (10 ml) was then added dropwise through an addition funnel within 5 min. The resulted mixture was stirred overnight at room temperature, and then filtered through Celite. The mother liquid was concentrated under vacuum, and the residue was submitted to purification with column chromatography using silica gel and solvent system from hexanes to 15/85 (v/v) ethyl acetate/hexanes, affording compound 16 as an off-white solid (1.105 g, 78%).¹H NMR (acetone-d⁶, ppm) δ 2.26 (s, 6H, C—H); 7.27 (s, 2H, Ar—H); 7.38 (s, 1H, N—H); 9.56 (s, 1H, O—H). ESI-TOF high accurate mass (M−H⁺) for C₁₁H₉N₄OCl₂, calculated, 283.016; found, 283.016.

Synthesis of DL-29: A solution of compound 16 (0.642 g, 2.25 mmol) in dimethylformamide (8 mL) was prepared first under nitrogen while stirring. This solution was then cooled to 0° C. on ice-water bath and water (0.5 mL) was added. To this solution, a solution of sodium hydrosulfide (0.566 g, 67%, 6.75 mmol) in water (1.0 mL) was added within 2 min. After the completion of the addition, the resulted mixture was stirred at 0° C. for 1 h, and 60° C. for 1 h. After cooling, the mixture was poured on to water (80 mL) and acidified with 3 N HCl to pH 3. The resulting precipitate was filtered, washed with water and dried in the air, affording DL-29 as an off-white solid (0.49 g, 78%). ¹H NMR (DMSO-d⁶, ppm) δ 2.17 (s, 6H, C—H); 7.04 (s, 2H, Ar—H); 8.34 (s, 1H, N—H); 8.87 (s, 1H, O—H); 12.10 (s, 1H, S—H); 13.02 (s, 1H, S—H). ESI-TOF high accurate mass (M+H⁺) for C₁₁H₁₃N₄OS₂, calculated: 281.053; found: 281.053.

Synthesis of compound 18: To a solution of cyanuric chloride (0.85 g, 4.62 mmol) in tetrahydrofuran (10 mL), was added anhydrous potassium carbonate (1.28 g, 9.24 mmol) under nitrogen, while stirring with a magnetic stirrer. To this suspension, 3-amino-4,6-di-tert-butylphenol (17, 1.022 g, 4.62 mmol) solution in tetrahydrofuran (10 ml) was then added dropwise through an addition funnel within 10 min. The resulted mixture was stirred overnight at room temperature, and then filtered through Celite. The mother liquid was concentrated under vacuum, and the residue was submitted to purification with column chromatography using silica gel and solvent system from hexanes to 10/90 (v/v) ethyl acetate/hexanes, affording compound 18 as a slightly pink solid (1.33 g, 78%).¹H NMR (acetone-d⁶, ppm) δ 1.31 (s, 9H, C—H); 1.45 (s, 9H, C—H); 6.73 (s, 1H, Ar—H); 7.41 (s, 1H, Ar—H); 8.46 (s, 1H, N—H); 9.19 (s, 1H, O—H). ESI-TOF high accurate mass (M−H⁺) for C₁₇H₂₁N₄OCl₂, calculated, 367.110; found, 367.110.

Synthesis of DL-30: A solution of compound 18 (0.716 g, 1.94 mmol) in dimethylformamide (6 mL) was prepared first under nitrogen while stirring. This solution was then cooled to 0° C. on ice-water bath and water (0.8 mL) was added. To this solution, a solution of sodium hydrosulfide (0.50 g, 67%, 5.82 mmol) in water (1.0 mL) was added within 2 min. After the completion of the addition, the resulted mixture was stirred at 0° C. for 1 h, and 60° C. for 1 h. After cooling, the mixture was poured on to water (80 mL) and acidified with 3 N HCl to pH 3. The resulting precipitate was filtered, washed with water and dried in the air, affording DL-30 as a white solid (0.69 g, 98%). ¹H NMR (DMSO-d⁶, ppm) δ 1.25 (s, 9H, C—H); 1.38 (s, 9H, C—H); 7.15 (s, 1H, Ar—H); 7.28 (s, 1H, Ar—H); 8.39 (s, 1H, O—H); 9.50 (s, 1H, N—H), 12.50 (s, 1H, S—H); 13.08 (s, 1H, S—H). ESI-TOF high accurate mass (M+H⁺) for C₁₇H₂₅N₄OS₂ (M+H⁺), calculated: 365.146; found: 365.147.

Synthesis of compound 19: To a solution of cyanuric chloride (0.416 g, 2.26 mmol) in tetrahydrofuran (5 mL), was added anhydrous potassium carbonate (0.624 g, 4.52 mmol) under nitrogen, while stirring with a magnetic stirrer. To this suspension, 2-amino-4,6-di-tert-butylphenol (19, 0.50 g, 2.26 mmol) solution in tetrahydrofuran (5 ml) was then added dropwise through an addition funnel within 10 min. The resulted mixture was stirred overnight at room temperature, and then filtered through Celite. The mother liquid was concentrated under vacuum, and the residue was submitted to purification with column chromatography using silica gel and solvent system from hexanes to 7.5/92.5 (v/v) ethyl acetate/hexanes, affording compound 20 as a white solid (0.83 g, 100%1).¹H NMR (acetone-d⁶, ppm) δ 1.31 (s, 9H, C—H); 1.45 (s, 9H, C—H); 7.26 (s, 1H, Ar—H); 7.35 (s, 1H, Ar—H); 7.70 (s, 1H, N—H); 9.29 (s, 1H, O—H). ESI-TOF high accurate mass (M−H⁺) for C₁₇H₂₁N₄OCl₂, calculated, 367.110; found, 367.110.

Synthesis of DL-31: A solution of compound 20 (0.783 g, 2.12 mmol) in dimethylformamide (7 mL) was prepared first under nitrogen while stirring. This solution was then cooled to 0° C. on ice-water bath and water (0.8 mL) was added. To this solution, a solution of sodium hydrosulfide (0.546 g, 67%, 6.36 mmol) in water (1.1 mL) was added within 2 min. After the completion of the addition, the resulted mixture was stirred at 0° C. for 1 h, and 60° C. for 1 h. After cooling, the mixture was poured on to water (80 mL) and acidified with 3 N HCl to pH 3. The resulting precipitate was filtered, washed with water and dried in the air, affording DL-31 as a white solid (0.753 g, 98%). ¹H NMR (DMSO-d⁶, ppm) δ 1.28 (s, 9H, C—H); 1.36 (s, 9H, C—H); 7.16 (s, 1H, Ar—H); 7.28 (s, 1H, Ar—H); 8.63 (s, 1H, N—H); 8.79 (s, 1H, N—H), 12.32 (s, 1H, S—H); 13.04 (s, 1H, S—H). ESI-TOF high accurate mass (M+H⁺) for C₁₇H₂₅N₄OS₂ (M+H⁺), calculated: 365.146; found: 365.148.

Synthesis of compound 22: To a solution of 2,6-dimethoxyphenol (21, 5.0 g, 31.79 mmol) in THE (160 mL), was added tert-butyl nitrite (12.6 mL). The resulted mixture was stirred at room temperature for 30 min. The mixture was then concentrated, and the residue was submitted to purification with column chromatography using silica gel and solvent system from hexanes to 15/85 (v/v) acetone/hexanes, affording compound 22 as a white solid (0.86 g, 14%).

Synthesis of compounds 23 and 24: To a solution of compound 22 (0.86 g, 0.43 mmol) in ethanol (100 mL), was added palladium on carbon (Pd/C, 10%, 0.86 g) under nitrogen. To this suspension, 1,4-cyclohexadiene (8.6 mL) was then added. The resulted mixture was stirred overnight (16 h) at room temperature. More 1,4-cyclohexadiene (5 mL) was then added, and the mixture was stirred for further 5 h at room temperature till the completion of the reduction. After the mixture was filtered through Celite, the mother liquid was concentrated in vacuum, affording compound 23, which is preserved in nitrogen atmosphere and used in next step without further purification.

To a solution of cyanuric chloride (0.794 g, 4.32 mmol) in THF (10 mL), was added anhydrous potassium carbonate (1.19 g, 8.64 mmol) under nitrogen, while stirring with a magnetic stirrer. To this suspension, compound 23 solution in tetrahydrofuran (10 ml) was then added dropwise within 10 min. The resulted mixture was stirred overnight at room temperature, and then filtered through Celite. The mother liquid was concentrated under vacuum, and the residue was submitted to purification with column chromatography using silica gel and solvent system from hexanes to 25/75 (v/v) ethyl acetate/hexanes, affording compound 24 as an off-white solid (0.587 g, 43% for two steps).¹H NMR (acetone-d⁶, ppm) δ 3.85 (s, 6H, C—H); 7.10 (s, 2H, Ar—H); 7.31 (s, 1H, N—H); 9.68 (s, 1H, O—H). ESI-TOF high accurate mass (M−H⁺) for C₁₁H₉N₄O₃Cl₂, calculated, 315.006; found, 315.006.

Synthesis of DL-32: A solution of compound 24 (0.491 g, 1.55 mmol) in DMF (5 mL) was prepared first under nitrogen while stirring. This solution was then cooled to 0° C. on ice-water bath and water (0.5 mL) was added. To this solution, a solution of sodium hydrosulfide (0.40 g, 67%, 4.65 mmol) in water (0.80 mL) was added within 2 min. After the completion of the addition, the resulted mixture was stirred at 0° C. for 1 h, and 60° C. for 1 h. After cooling, the mixture was poured on to water (80 mL) and acidified with 3 N HCl to pH 3. The resulting precipitate was filtered, washed with water and dried in the air, affording DL-32 as an off-white solid (0.449 g, 93%). ¹H NMR (DMSO-d⁶, ppm) δ 3.75 (s, 6H, C—H); 6.85 (s, 2H, Ar—H); 8.45 (s, 1H, N—H); 9.06 (s, 1H, O—H), 12.17 (s, 1H, S—H); 13.05 (s, 1H, S—H). ESI-TOF high accurate mass (M+H⁺) for C₁₁H₁₃N₄O₃S₂, calculated: 313.042; found: 313.043.

Synthesis of compound 26: To a solution of tetraethylene glycol (25, 5.4 g, 27.8 mmol) in THE (40 mL), was added sodium hydride (1.2 g) in portions at −10° C. To the suspension, propargyl bromide (2.96 mL) was added dropwise at this temperature. The resulted mixture was allowed to warm to room temperature and stirred at this temperature overnight. The mixture was filtered through Celite, concentrated in vacuum and the residue was submitted to purification with column chromatography using silica gel and solvent system from dichloromethane to 3/97 (v/v) methanol/dichloromethane, affording compound 26 as a colorless oil (2.7 g, 42%).

Synthesis of compound 27: To a solution of compound 26 (2.7 g, 11.6 mmol) in dichloromethane (5 mL), was added triethylamine (3.2 mL, 23.2 mmol). The resulted mixture was cooled to 0° C. To the mixture, tosyl chloride (2.33 g, 12.2 mmol) was added. The resulted mixture was allowed to warm to room temperature and stirred at room temperature overnight. To the mixture, water (10 mL) was added. The resulted mixture was extracted with dichloromethane (3×10 mL). The organic layer was washed with 6 N HCl (15 mL), water (10 mL), sodium bicarbonate (5%, 10 mL) and brine (10 mL), dried over magnesium sulfate and concentrated in vacuum. The residue was submitted to purification with column chromatography using silica gel and solvent system from dichloromethane to 2/98 (v/v) methanol/dichloromethane, affording compound 27 as a colorless oil (3.83 g, 86%).

Synthesis of compound 28: To a solution of compound 9 (1.72 g, 4.9 mmol) in acetonitrile (20 mL), were added potassium carbonate (3.48 g, 25.2 mmol) and compound 27 (1.88 g, 4.9 mmol) under nitrogen. The resulted mixture was refluxed on an oil bath overnight. After cooling to room temperature, the mixture was filtered through Celite. The mother liquid was concentrated in vacuum. The residue was submitted to purification with column chromatography using silica gel and solvent system from hexanes to 10/90 (v/v) acetone/hexanes, affording compound 28 as a white solid (0.91 g, 33%). ¹H NMR (acetone-d⁶, ppm) δ 1.49 (s, 18H, C—H); 2.92 (t, 1H, J=4 Hz, CC—H); 3.62-3.69 (m, 10H, C—H); 3.72-3.73 (m, 2H, C—H); 3.92-3.94 (m, 2H, C—H); 4.01-4.02 (m, 2H, C—H); 4.20 (d, 2H, J=4 Hz, C—H); 7.43 (s, 2H, Ar—H); 7.93-7.95 (m, 4H, Ar—H). ESI-TOF high accurate mass (M+Na⁺) for C₃₃H₄₃NO₇Na, calculated, 588.293; found, 588.293.

Synthesis of compounds 29 and 30: To a solution of compound 28 (1.40 g, 2.48 mmol) in acetonitrile (40 mL) and water (23 mL), was added hydrazine (65%, 1.5 mL). The resulted mixture was stirred at room temperature for 2 h. To the mixture, sodium hydroxide solution (5%, 7 mL) was added. The resulted mixture was stirred overnight. To the mixture, water (100 mL) was added. The resulted mixture was extracted with ethyl acetate (3×30 mL). The organic layer was washed with water and brine consecutively, dried over magnesium sulfate and concentrated in vacuum, affording compound 29 (1.01 g, 94%), which is being used in next step without further purification. ¹H NMR (acetone-d⁶, ppm) δ 1.39 (s, 9H, C—H); 1.43 (s, 9H, C—H); 2.92 (t, 1H, J=4 Hz, CC—H); 3.60-3.70 (m, 12H, C—H); 3.87-3.99 (m, 4H, C—H); 4.19 (d, 2H, J=4 Hz, C—H); 6.57 (s, 2H, Ar—H); 6.61 (s, 2H, N—H). ESI-TOF high accurate mass (M+H⁺) for C₂₅H₄₂NO₅, calculated, 436.306; found, 436.306.

To a solution of cyanuric chloride (0.424 g, 2.30 mmol) in THF (5 mL), was added anhydrous potassium carbonate (0.635 g, 4.6 mmol) under nitrogen, while stirring with a magnetic stirrer. To this suspension, a solution of compound 29 in tetrahydrofuran (8 ml) was then added dropwise through an addition funnel within 20 min. The resulted mixture was stirred overnight at room temperature, and then filtered through Celite. The mother liquid was concentrated under vacuum, and the residue was submitted to purification with column chromatography using silica gel and solvent system from dichloromethane to 15/85 (v/v) ethyl acetate/dichloromethane, affording compound 30 as a white solid (1.32 g, 98%). ¹H NMR (acetone-d⁶, ppm) δ 1.47 (s, 18H, C—H); 2.92 (t, 1H, J=4 Hz, CC—H); 3.56-3.73 (m, 12H, C—H); 3.90-4.09 (m, 4H, C—H); 4.19 (d, 2H, J=4 Hz, C—H); 7.72 (s, 2H, Ar—H); 9.80 (s, 1H, N—H). ESI-TOF high accurate mass (M+H⁺) for C₂₈H₄₁N₄O₅Cl₂, calculated, 583.245; found, 583.245.

Synthesis of DL-33: A solution of compound 30 (1.32 g, 2.26 mmol) in DMF (4 mL) was prepared first under nitrogen while stirring. This solution was then cooled to 0° C. on ice-water bath and water (0.5 mL) was added. To this solution, a solution of sodium hydrosulfide (0.60 g, 67%, 4.65 mmol) in water (1.2 mL) was added within 2 min. After the completion of the addition, the resulted mixture was stirred at 0° C. for 1 h, and 60° C. for 1 h. After cooling, the mixture was poured on to water (50 mL) and acidified with 3 N HCl to pH 3. The resulting precipitate was filtered, washed with water and dried in the air, affording DL-33 as a white solid (1.13 g, 86%). ¹H NMR (DMSO-d⁶, ppm) δ 1.44 (s, 18H, C—H); 2.92 (t, 1H, J=4 Hz, CC—H); 3.56-3.73 (m, 12H, C—H); 3.88-4.01 (m, 4H, C—H); 4.20 (d, 2H, J=4 Hz, C—H); 7.69 (s, 2H, Ar—H); 9.20 (s, 1H, N—H), 11.53 (s, 1H, S—H). ESI-TOF high accurate mass (M+H⁺) for C₂₈H₄₃N₄O₅S₂, calculated: 579.267; found: 579.267.

Measurement of the Extinction Coefficients of the Cholesterol Lowering Compounds:

The compounds were dissolved in DMSO to either 50 mg/ml, 10 mg/ml or 5 mg/ml solutions, which were diluted to 0.5 mg/ml with DMSO. These solutions were then diluted to 5 ug/ml with 1 x PBS buffer. The resulted solutions were then measured in a UV-vis spectrophotometer for UV scanning from 240 nm to 400 nm at 25° C. The absorbance values were subtracted by that of a blank, 1 x PBS buffer containing DMSO. The total volume of the solutions was 300 uL and the path length for each well was 0.7 cm in a UV 96-well plate. The extinction coefficients of the compounds were then calculated based on molar concentrations and the corrected UV absorbances at the maximum absorption (Table 1).

Measurement of the solubilities of cholesterol lowering compounds in water and phosphate buffer: Mixing of cholesterol lowering compounds with either water or a phosphate buffer (50 mM, pH 7) separately, resulted in individual mixtures. The mixtures wer stirred for 1 h at room temperature, transferred to 1.5 mL Eppendorf tubes, and centrifuged for 2 min. The clear solutions were filtered through filter papers, affording clear solutions, which were centrifuged again for 2 min. The top clear solutions were diluted by 1 x PBS buffer by 10-fold for the solubilities in water and 100-fold for the solubilities in 1 x PBS buffer. The new clear solutions were then measured individually using a UV-vis spectrophotometer and a UV 96-well microplate. The absorbance values were subtracted by that of a blank, 1 x PBS buffer. Concentrations and solubilities of the compounds were then calculated using their extinct coefficients and the corrected UV absorbances at the maximum absorptions (Table 1).

### Example 12

### Biological Assays

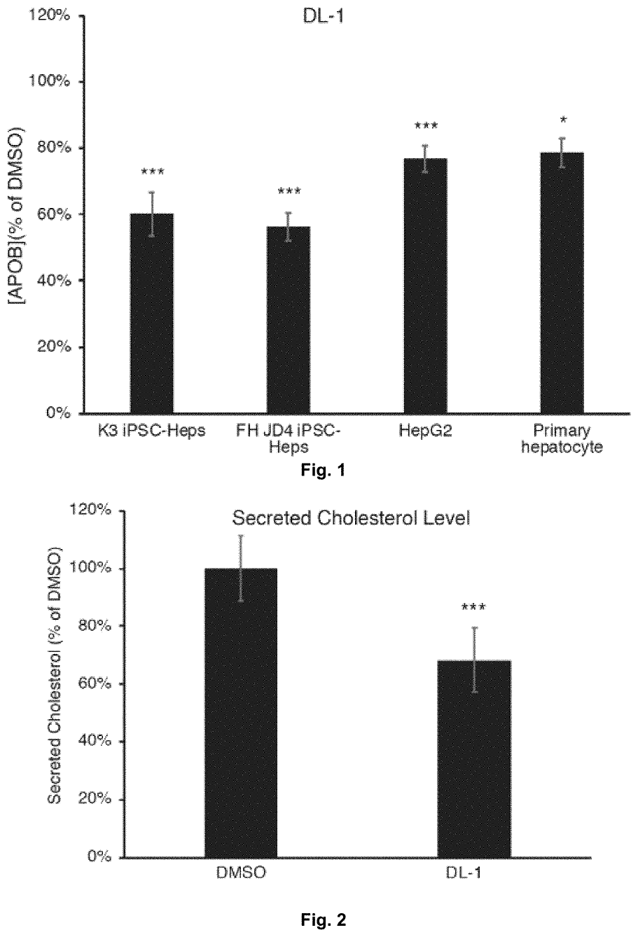

A. Human-induced pluripotent stem cells from a health donor (K3) and HoFH patient (JD4) were differentiated into hepatocytes via a defined protocol. Two different iPSC-derived Hepatocytes along with human primary hepatocytes and HepG2 cells were treated with 2 μg/mL of DL-1 for 24 hours and secreted APOB level were determined via human-specific APOB ELISA and were compared to DMSO-treated cells. As shown in FIG. 1, DL-1 reduces APOB secretion in the culture medium of hepatic cells. Data shown as mean±SEM of triplicates (n=3); * p<0.05, *** P<0.001.

B. Human iPSC-derived hepatocytes were treated with 2 μg/mL of DL-1 for 24 hours. Total cholesterol levels within the medium were determined and normalized with DMSO treated control. Data are shown as mean±SEM of six replicates (n=6); * ** P<0.001. As shown in FIG. 2, DL-2 reduced total cholesterol level in culture medium of hepatic cells.

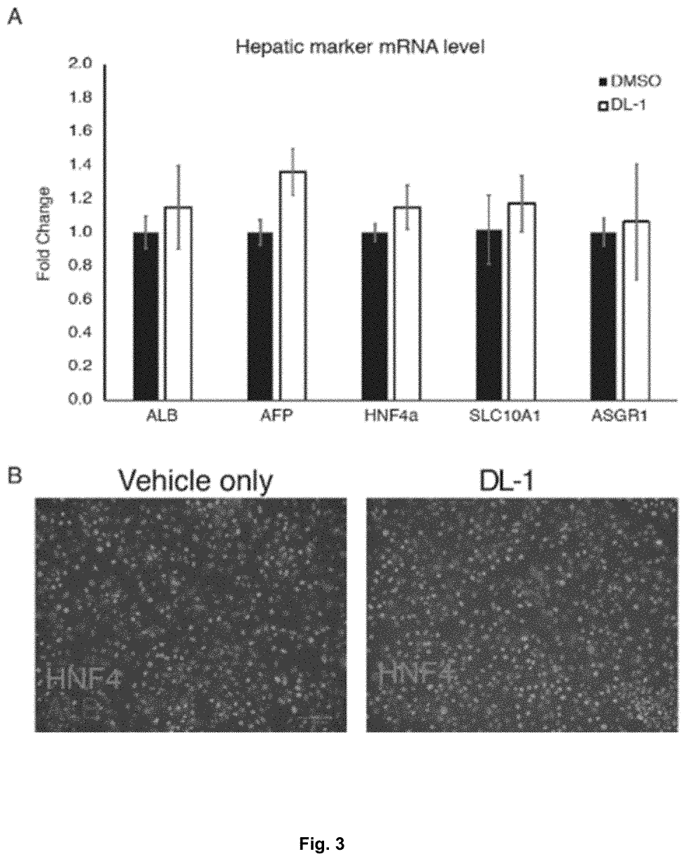

C. Human iPSC-derived hepatocytes were treated with 2 μg/mL of DL-1 for 24 hours. (A) mRNA level of hepatic markers (ALB, AFP, HNF4α, SLC10A1, ASGR1) were determined via real-time qPCR. (B) Cells were fixed and immunofluorescence was performed to determine HNF4 and ALB level. FIG. 3 shows that DL-1 has no impact on hepatic characters. Data are shown as mean SEM of six replicates (n=4).

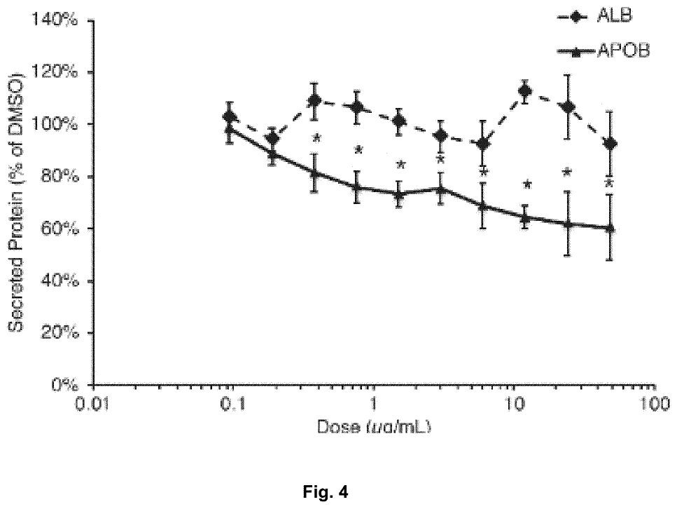

D. Human iPSC-derived hepatocytes were treated with DL-1 with concentrations ranging from 0.0325 to 12 or 48 μg/mL for 24 hours. Pre-drug and Post-drug medium were collected for analysis. Albumin and APOB levels were determined via ELISA assay. All secreted proteins were compared to non-treated cells. FIG. 4 shows that DL-1 reduces APOB secretion level in a dose-response manner without affecting albumin. Data are shown as mean±SEM of four replicates (n=4); * p<0.01.

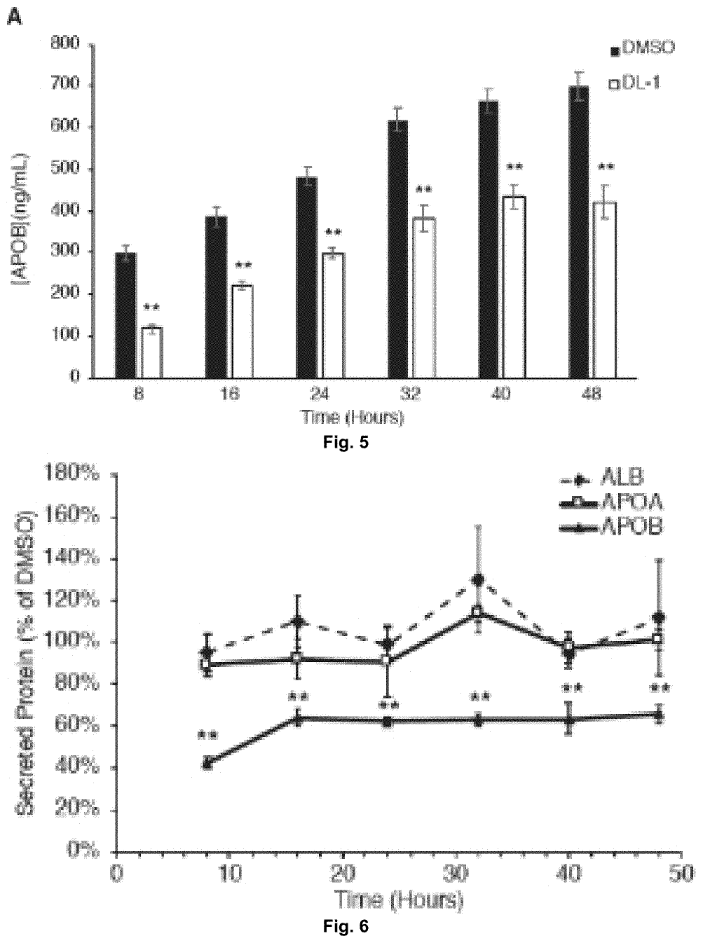

E. Human iPSC-derived hepatocytes were treated with 2 μg/mL of DL-1 from 8 to 48 hours. Albumin, APOPA, and APOB level in the culture medium were determined via ELISA. All secreted proteins were compared to non-treated cells. In FIG. 5, accumulated APOB in the medium from different time-point was measured and normalized to albumin level. In FIG. 6, APOA, APOB, and albumin in the medium at the indicated times were compared to control. Thus, FIGS. 5 and 6 show that DL-1 reduces APOB level in a time-dependent manner without affecting albumin and APOA level. Data are shown as mean±SEM of four replicates (n=4); *p<0.05, **p<0.01.

F. Twenty-one compounds that are structurally similar to DL-1 were tested with the concentration of 2 μg/mL in human iPSC-derived hepatocytes for 24 hours. Secreted APOB levels were determined and compared to the DMSO-treated group. FIG. 9 demonstrates that DL-1 derivatives show APOB reduction in human iPSC-derived hepatocytes. Data is shown as mean±SEM of replicates (n=8) and as quantification value of each derivative.

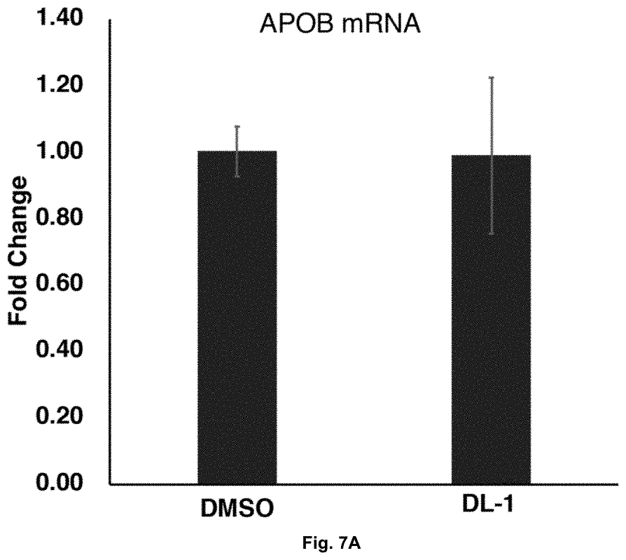

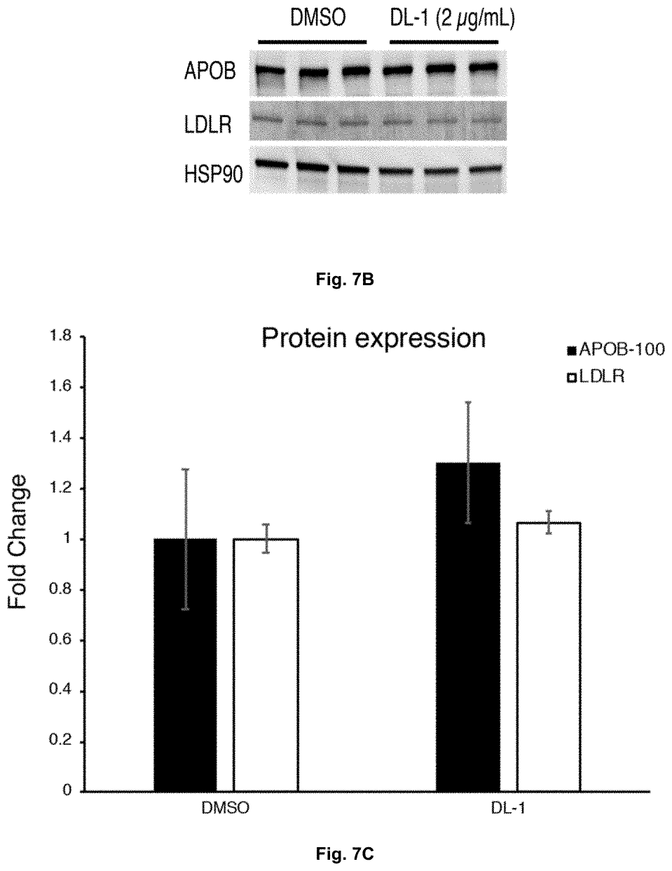

G. Human iPSC-derived hepatocytes were treated with 2 μg/mL of DL-1 or equivalent concentration of DMSO for 24 hours. FIG. 7 shows that DL-1 has no impact on APOB mRNA and intracellular protein levels. (A) APOB mRNA level was determined using real-time qPCR. (B) Intracellular APOB, LDLR, and HSP90 protein levels were determined via immunoblot. (C) Quantification of biological replicates of immunoblots on samples treated as in (B). Data are shown as mean±SEM of four replicates (n=4).

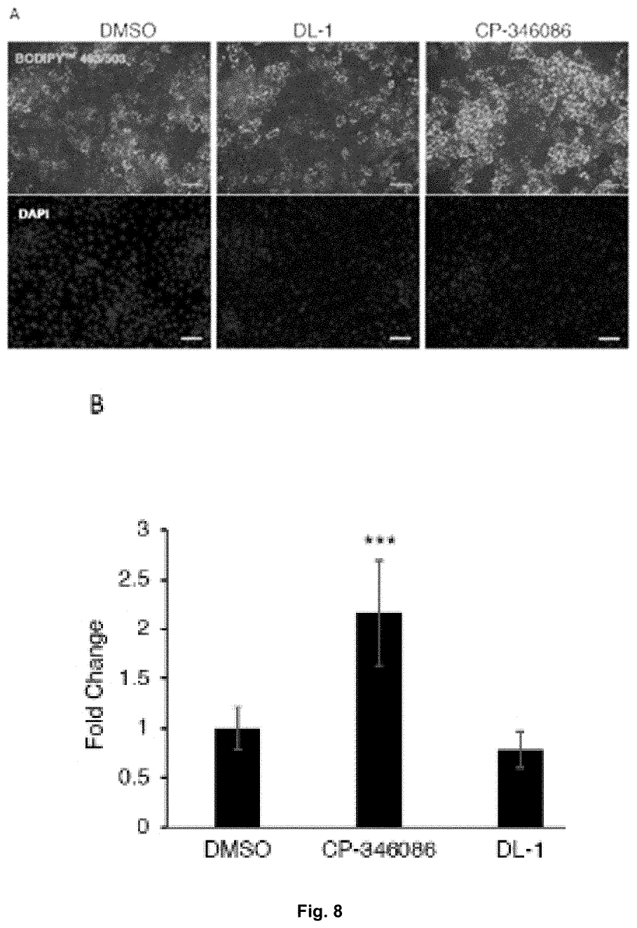

H. Human iPSC derived hepatocytes were treated with DL-1 (2 μg/mL) and MTTP inhibitor CP-346086 (20 nM) for 72 hours. FIG. 8 shows that DL-1 has no impact on hepatic lipid accumulation comparing to MTTP inhibitor. (A) Hepatic neutral lipid was determined by staining with 2 μM of BODIPYTM 493/503 for 15 mins and fixed with 4% PFA for microscopy. Nuclei were stained blue by DAPI. Scale bar=100 μm. (B) Bar graph showing a significant increase of neutral lipid level was observed in response to CP-346086. Data are shown as mean±SEM of ten replicates (n=10); ***p<0.001.

I. Human induced pluripotent stem cells (iPSCs) were differentiated to hepatocyte-like cells using a defined culture medium, conditions, and growth factors as described in Si-Tayeb, K. et al. Highly efficient generation of human hepatocyte-like cells from induced pluripotent stem cells. Hepatology. 51 (1), 297-305 (2010).

Differentiated human hepatocyte-like cells were treated with representative compounds of the invention (2 μg/mL) or equivalent amount of DMSO (0.5%) as the placebo group for 24 hours. Culture medium before or after compound treatment were harvested for analysis. Pre- and Post-drug medium were both incubated exactly 24 hours in order to compare the differences in secreted LDL-C level in the same period of time. The secreted level of apolipoprotein B (APOB-100), the core protein of LDL-C, were determined via enzyme-linked immunosorbent assay (ELISA).

The post-drug: pre-drug ratio of APOB was determined for each compound and was compared to the placebo group. The experiment was performed in three biological replicates and the following data was obtained:

| | APOB | | |
|---|---|---|---|
| Structure | reduction | S.D | P value |
| 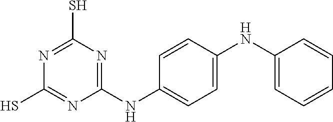 [SMILES: Sc1nc(S)nc(Nc2ccc(Nc3ccccc3)cc2)n1] | 39.80 | 6.55 | 0.00011868 |
| 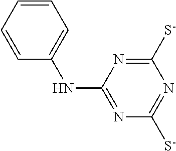 [SMILES: [S-]c1nc([S-])nc(Nc2ccccc2)n1] | 13.55 | 5.25 | 0.005227147 |
| 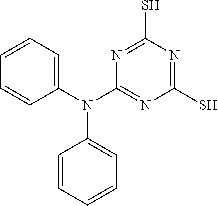 [SMILES: Sc1nc(S)nc(N(c2ccccc2)c2ccccc2)n1] | 20.22 | 3.43 | 1.76868E−05 |
| 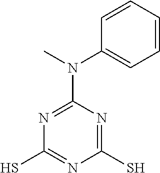 [SMILES: CN(c1ccccc1)c1nc(S)nc(S)n1] | 16.15 | 2.66 | 2.48239E−05 |
| 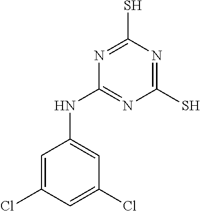 [SMILES: Sc1nc(S)nc(Nc2cc(Cl)cc(Cl)c2)n1] | 45.47 | 4.69 | 1.31365E−06 |
| 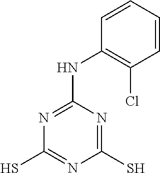 [SMILES: Sc1nc(S)nc(Nc2ccccc2Cl)n1] | 21.44 | 3.25 | 6.75563E−06 |
| 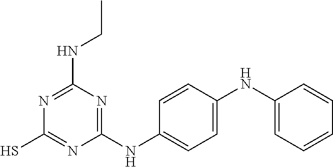 [SMILES: CCNc1nc(S)nc(Nc2ccc(Nc3ccccc3)cc2)n1] | 26.02 | 9.17 | 0.006086145 |
| 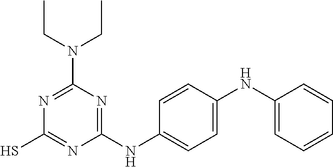 [SMILES: CCN(CC)c1nc(S)nc(Nc2ccc(Nc3ccccc3)cc2)n1] | 40.13 | 6.52 | 0.000108782 |
| 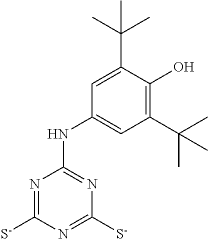 [SMILES: CC(C)(C)c1cc(Nc2nc([S-])nc([S-])n2)cc(C(C)(C)C)c1O] | 46.72 | 1.32 | 9.46706E−09 |
| 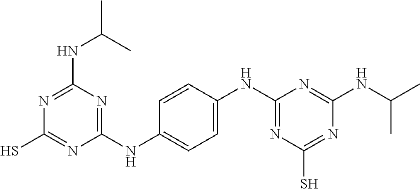 [SMILES: CC(C)Nc1nc(S)nc(Nc2ccc(Nc3nc(S)nc(NC(C)C)n3)cc2)n1] | 59.49 | 1.13 | 4.37835E−10 |
| 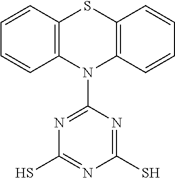 [SMILES: Sc1nc(S)nc(N2c3ccccc3Sc3ccccc32)n1] | 28.51 | 7.49 | 0.001446659 |
| 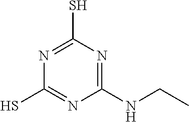 | 8.25 | 9.84 | 0.194086767 |
| 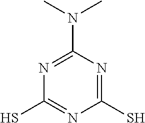 | 14.16 | 2.85 | 0.000106703 |
| 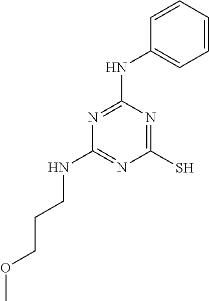 [SMILES: COCCCNc1nc(S)nc(Nc2ccccc2)n1] | 23.52 | 10.72 | 0.016267779 |
| 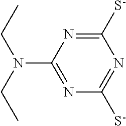 | 0.00 | 5.64 | 0.873790601 |
| 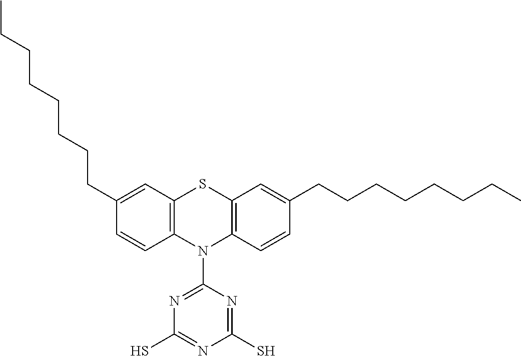 [SMILES: CCCCCCCCc1ccc2c(c1)Sc1cc(CCCCCCCC)ccc1N2c1nc(S)nc(S)n1] | 8.91 | 6.76 | 0.086890068 |
| 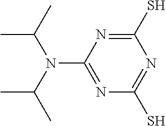 | 10.21 | 4.80 | 0.025402552 |
| 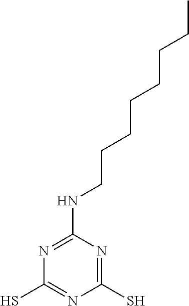 [SMILES: CCCCCCCCNc1nc(S)nc(S)n1] | 27.86 | 2.84 | 1.27004E−05 |
| 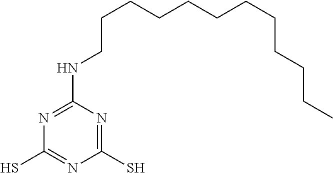 [SMILES: CCCCCCCCCCCCNc1nc(S)nc(S)n1] | 30.38 | 1.94 | 9.35854E−06 |
| 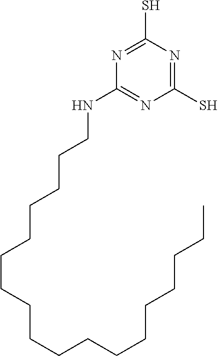 [SMILES: CCCCCCCCCCCCCCCCCCNc1nc(S)nc(S)n1] | 31.53 | 8.01 | 0.000554469 |
| 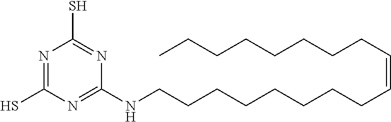 [SMILES: CCCCCCCC/C=C\CCCCCCCCNc1nc(S)nc(S)n1] | 34.84 | 2.16 | 2.75517E−06 |
| 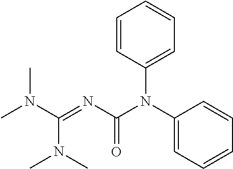 [SMILES: CN(C)C(=NC(=O)N(c1ccccc1)c1ccccc1)N(C)C] | increase 3.3 | 3.56 | 0.367521792 |
| 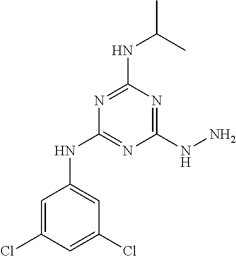 [SMILES: CC(C)Nc1nc(NN)nc(Nc2cc(Cl)cc(Cl)c2)n1] | Increase 28 | 7.49 | 0.000563386 |
| 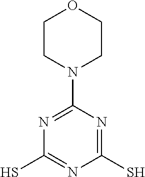 [SMILES: Sc1nc(S)nc(N2CCOCC2)n1] | Increase 10.8 | 8.12 | 0.074301277 |
| 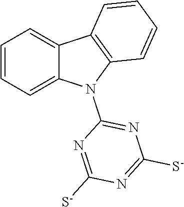 [SMILES: [S-]c1nc([S-])nc(-n2c3ccccc3c3ccccc32)n1] | 34.98 | 5.26 | 9.22284E−06 |
| 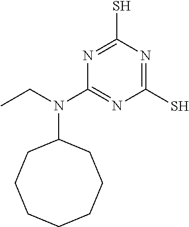 [SMILES: CCN(c1nc(S)nc(S)n1)C1CCCCCCC1] | 15.56 | 12.71 | 0.0851143 |

### Example 13

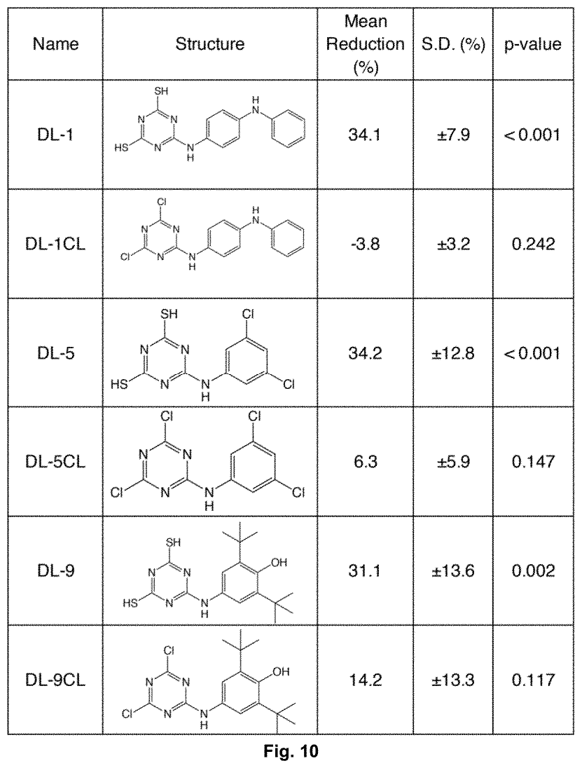

A. Thiol groups on the triazine structure from DL-1, DL-5, and DL-9 were substituted with chlorine to form DL-1CL, DL-5CL, and DL-9CL. Human iPSC-derived hepatocytes were then treated with 2 ag/mL of the newly synthesized compounds for 24 hours. Secreted APOB levels were determined and compared to the DMSO-treated group. FIG. 10 shows that thiol group help reduce hepatic APOB secretion. Data are shown as mean±SEM of replicates (n=4). Statistical analysis was performed using the student t-test.

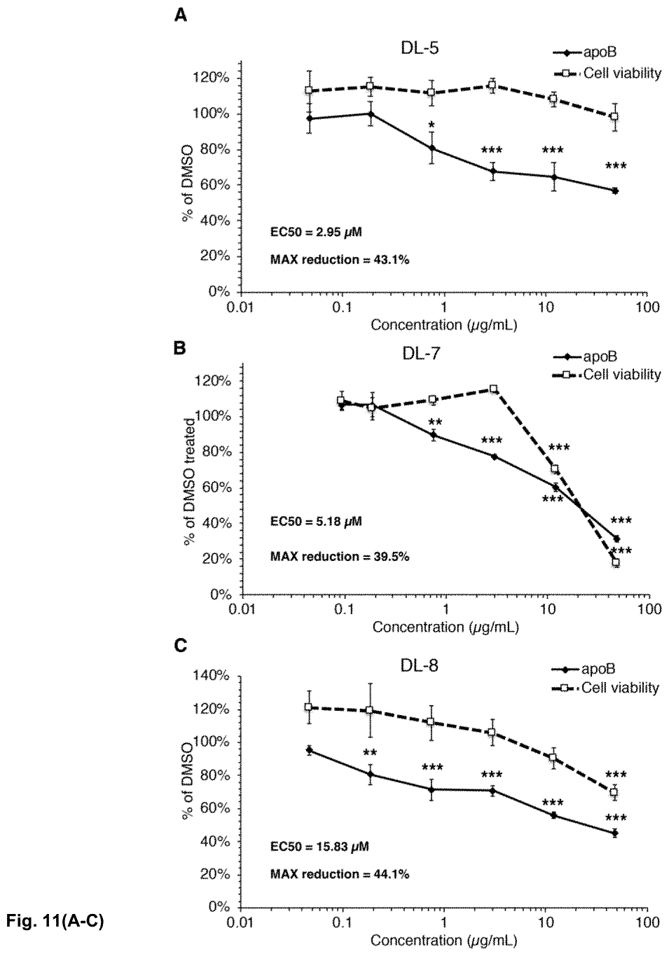

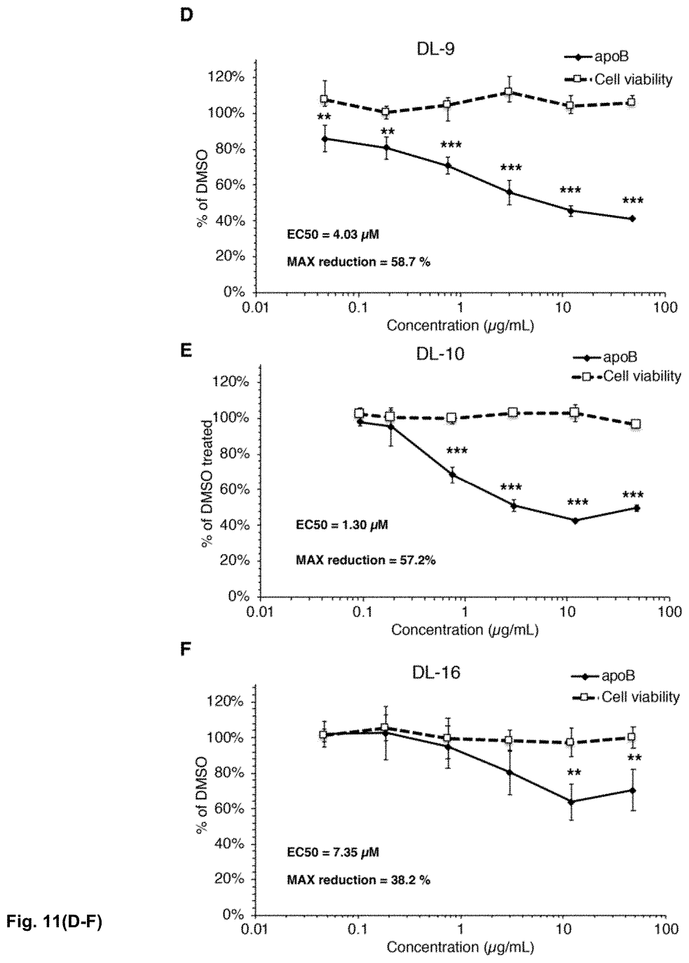

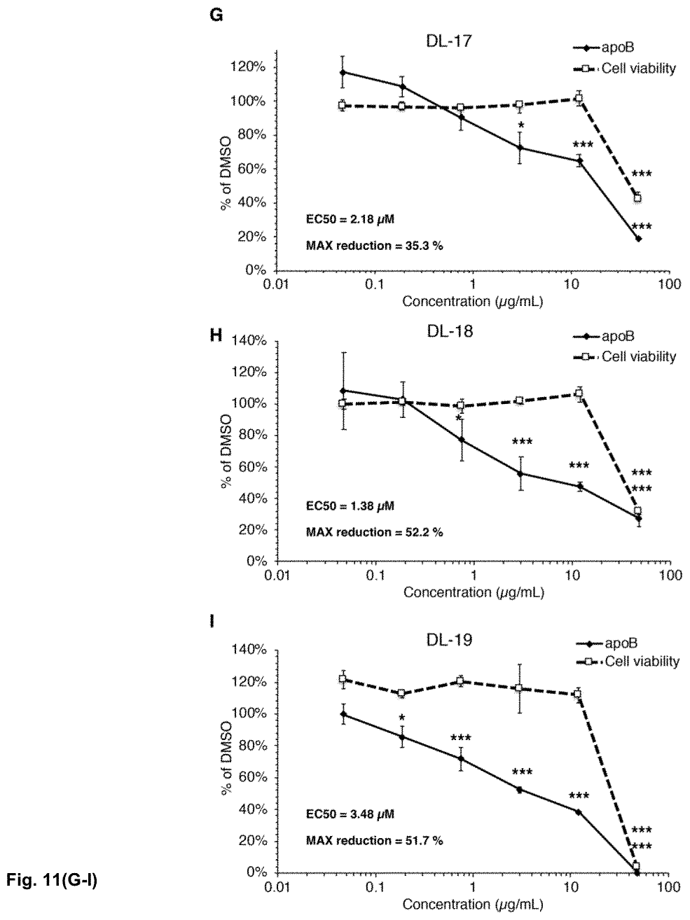

B. Human iPSC-derived hepatocytes were treated with DL-1 derivatives, which reduced more than 20% of APOB secretion in the single-dose (2 μg/mL) treatment. Cells were treated with various doses up to 48 μg/mL for 24 hours. Pre-drug and Post-drug medium were collected, and APOB levels were tested via ELISA assay. Post-drug: Pre-drug ratio was determined and normalized with DMSO treated group. Cell viability was determined using the ATP-based luminescent assay. FIG. 11 shows a dose-response analysis of DL-1 derivatives. Data are shown as mean±SEM of ten replicates (n=10); *p<0.05, **p<0.01, ***p<0.001.

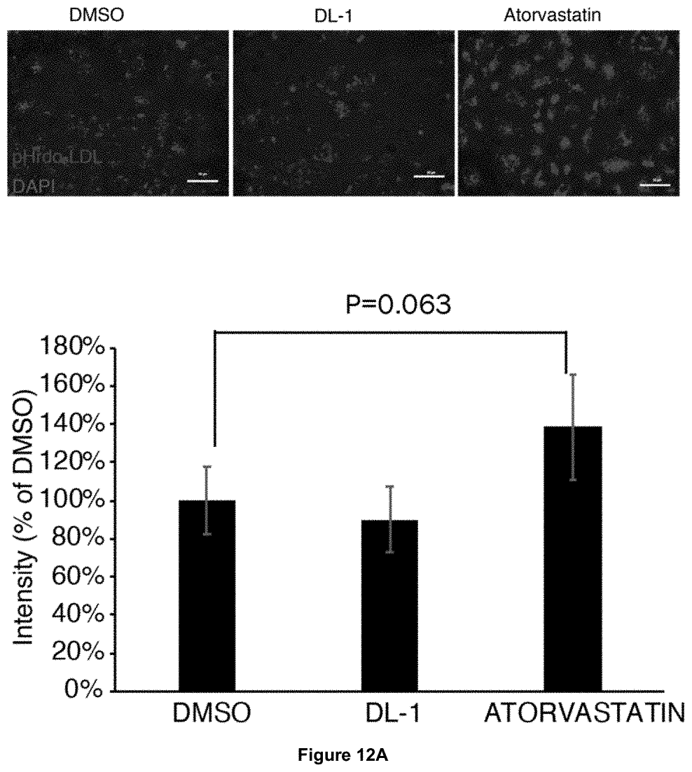

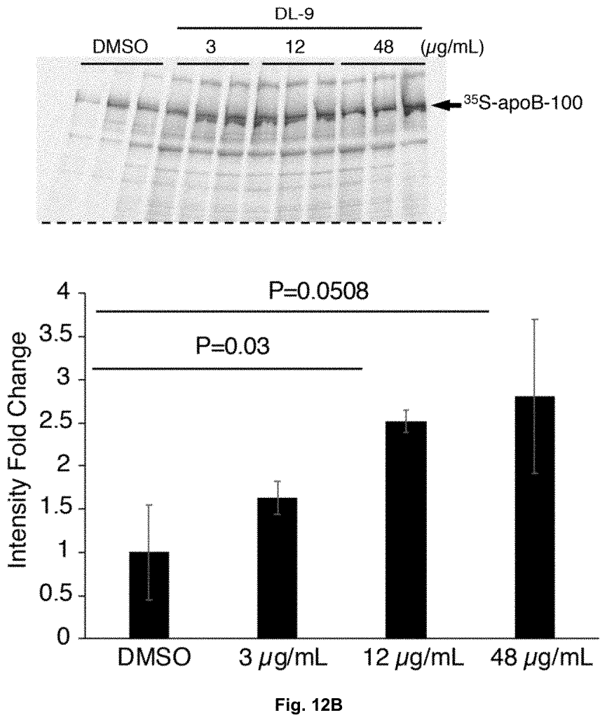

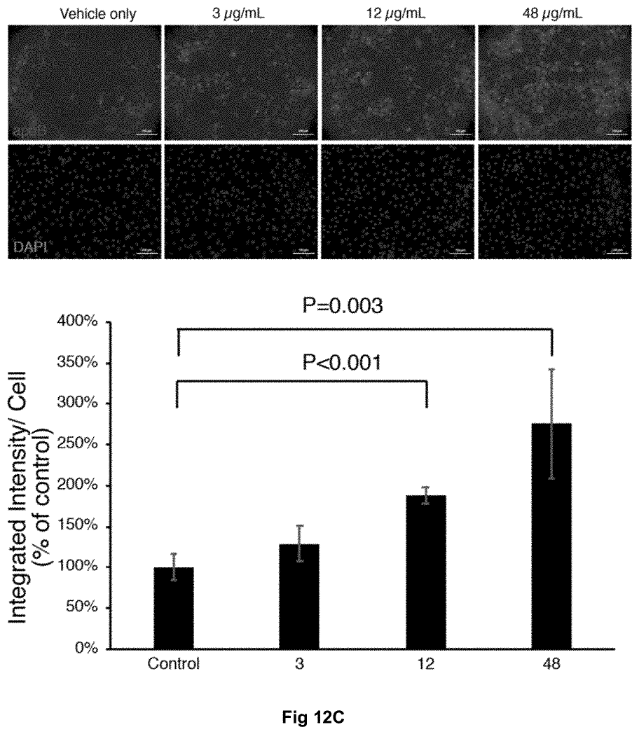

C. Human iPSC-derived hepatocytes (iPSC-Heps) were treated with 2 μg/mL of DL-1 or equivalent concentration of DMSO for 24 hours. In FIG. 7A, APOB mRNA level was determined using real-time qPCR. In FIG. 7B Intracellular APOB, LDLR, and HSP90 protein levels were determined via immunoblot; Quantification of biological replicates of immunoblots. Data are shown as mean±SD of four replicates (n=4). In FIG. 12A LDL uptake assay was performed by pre-treating cells with 2 μg/mL of DL-1, 20 μM of Atorvastatin, and equivalent concentration of DMSO for 24 hours followed by 4 hours of pHrodo-LDL exposure; LDL endocytosis was determined and quantified by fluorescence image; data are shown as mean±SD of triplicates (n=3). In FIG. 12B, iPSC-Heps were pre-treated with 3, 12, and 48 μg/mL of DL-9 for 2 hours, and cells were pulse-labeled with [³⁵S]methionine for 10 minutes and chased for 120 mins with or without DL-9 treatment; intracellular ³⁵S-APOB were determined by immunoprecipitation using anti-APOB antibody; band density was quantified and normalized with 5% of input (n=3). In FIG. 12C, immunofluorescence for APOB was detected by a specific anti-APOB antibody; bar graph showed integrative fluorescence intensity was quantified and normalized with cell numbers detected by DAPI staining (n=5). FIGS. 7 and 12 show that DL-1 and derivatives induced intracellular APOB accumulation independently of mRNA expression and LDL uptake. Data is shown as mean±SD. Statistical analysis was performed using student t-test.

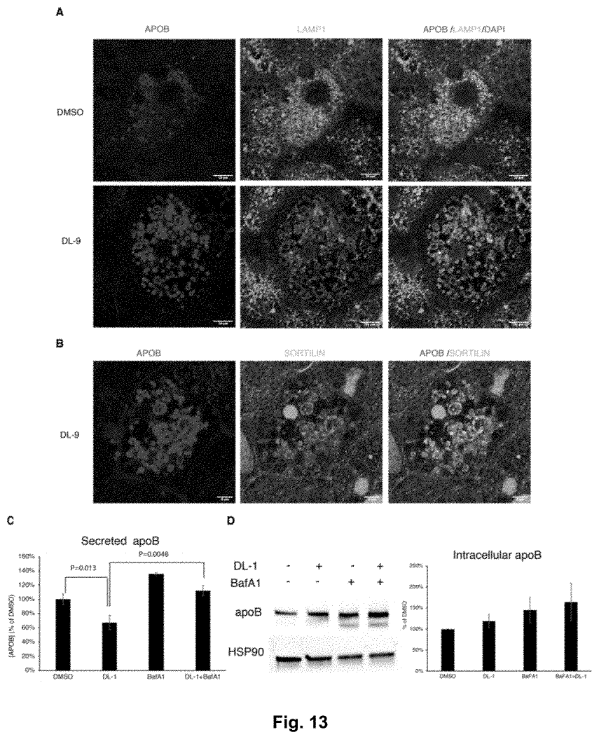

D. FIG. 13 shows that compounds of the present invention induced presecretory intracellular APOB aggregation and lysosomal degradation. Human iPSC-derived hepatocytes (iPSC-Heps) were treated with 48 μg/mL of DL-9 for 24 hours. Immunofluorescence of intracellular APOB, LAMP1, and SORTILIN was detected by specific antibodies. Double confocal immunofluorescence shows colocalization of APOB and lysosome marker LAMP1 in cells with or without 48 μg/mL DL-9 treatment (FIG. 13A). Double confocal immunofluorescence shows colocalization of APOB and SORTILIN in DL-9-treated cells; human iPSC-Heps were co-treated with 100 nM lysosomal inhibitor BafA1 and 2 μg/mL DL-1 for 24 hours (FIG. 13B). Extracellular APOB was measured by ELISA analysis. APOB level was normalized to DMSO treated group (FIG. 13C). In FIG. 13D intracellular APOB was determined via western blot using a specific anti-APOB antibody. The bar graph showed the quantification of the band intensity after normalized to housekeeping protein. Data are shown as mean±SD of triplicates (n=3). Statistical analysis was performed using the student t-test.

Induced Pluripotent Stem Cell and Hepatic Differentiation

Human iPSCs were generated previously and have been described elsewhere (Si-Tayeb, 2010; Cayo et al., 2012). K3 iPSCs were produced from foreskin fibroblasts (CRL2097) obtained from ATCC, and JD4 iPSCs were produced from primary dermal fibroblasts (GM02408) from a familial hypercholesterolemia patient that was provided by the Coriell Institute for Medical Research (Coriell Cell Repositories, Camden, N.J.) (Cayo et al., 2012). The hepatic differentiate protocol has been published (in Si-Tayeb 2010, Mallanna 2013, and Liu 2018) with minor modification. Briefly, human iPSCs were seeded on Matrigel-coated tissue culture plates with the density of 7×10′/mL to form a monolayer. Cells were induced to form definitive endoderm by the addition of 100 ng/ml Activin A for 5 days. Definitive endoderm was then converted to hepatic progenitor cells by addition of Bone Morphogenetic Protein 4 (20 ng/ml) and Fibroblast Growth Factor 2 (10 ng/ml) for an additional 5 days. Immature hepatocytes were generated by the inclusion of Hepatocyte Growth Factor (20 ng/ml). Cells were induced to mature hepatocyte-like cells by the addition of Oncostatin M (20 ng/ml) for 5 days.

Drug Screen and Compound Library

K3 iPSCs were differentiated to hepatocyte-like cells in 96-well plates. A 24 hours pre-drug treatment sample of the medium was collected before the addition of drugs from the South Carolina Compound Collection library (representative set) for 24 hours at 2 μg/mL, and post-drug medium were harvested for analysis. APOB levels in the collected medium were determined using a standard curve, and Four Parameter Logistic (4PL) regression model in both pre- and post-drug treated samples by ELISA. The APOB levels in the pre-drug and post-drug medium were combined and expressed as a delta-APOB ratio (post-drug [APOB]:pre-drug [APOB]). A z-score was generated for each individual compound using the delta-APOB ratio with the standard deviation of the delta-APOB ratios. Primary hits were validated in secondary replicate experiments (biological replicates n=4), and statistical significance was determined by a Student's t-test. The South Carolina Compound Collection (SC3) library consists of 130,000 proprietary drug-like small molecules. The average physicochemical properties of SC3 are MW˜400 g/mol, cLogP˜2, and ˜7 rotatable bonds. In terms of diversity, SC3 has more than 10,000 clusters with a threshold of 50% similarity and ˜70,000 clusters at 80% similarity. SC3 representative set consists of 10,000 small molecules that represent 10,000 clusters of compounds from the SC3 library.

Enzyme-Linked Immunosorbent Assays (ELISA)

Human APOB level was determined via a commercial sandwich ELISA (3715-1H-6; Mabtech), and the signal was detected using the HRP-TMB system. The concentration of APOB was determined using a standard curve with four parameters logistic regression model. Human APOA level was determined via a commercial sandwich ELISA (3710-1H-6; Mabtech) following manufacture instruction. Human albumin levels in cell culture supernatants were measured by commercial ELISA (E88-129; Bethyl Laboratories) following manufactural instructions.

Metabolic Labeling, Immunoprecipitation, and Immunoblot

Human iPSC-derived hepatocytes were pretreated with 3, 12, and 48 μg/mL of DL-9 for 2 hours. Cells were then starved with methionine-free DMEM medium for 30 mins before pulse-labeling with [35S]-Met DMEM medium for 10 mins. The medium was replaced with growth medium with 3, 12, and 48 μg/mL of DL-9 for 2 hours, and cell lysates were collected.

Immunoprecipitation of cell lysates was performed using protein G magnet beads (ThermoFisher) and anti-APOB (LDL20/17, Mabtech) antibodies. Eluted protein samples were separated using 4-15% acrylamide gradient SDS-PAGE. The radio-labeled signal was developed using FLA 9000 Typhoon Storage Phosphorimager (GE Healthcare). Expression levels were quantified via integrated intensity normalized by 5% of input of each sample. For western blot, human iPSC-derived hepatocytes were treated with 4 μg/mL of BL-1 for 24 hours. Protein concentrations were determined by BCA assay (Bio-Rad), and 20 μg of protein was used for SDS-PAGE separation followed by PVDF membranes transfer. Membranes were probed with anti-APOB (LDL 20/17, Mabtech), LDLR(C3) (sc-18823, Santa Cruz), and HSP90 (ab32568, Abcam). Signals were detected using HRP-conjugated secondary antibodies. Protein expression was quantified via the stain-free imaging system (Bio-Rad) and was normalized to total protein using image Lab software (Bio-Rad)(Colella 2012).

Immunostaining

For hepatic marker expression analysis, human iPSC-derived hepatocytes (Day 20) were treated with 2 μg/mL of DL-1 for 24 hours. Cells were fixed in 4% paraformaldehyde (PFA) and penetrated with 0.1% Triton-X-100. Cells were incubated with primary antibodies (ALB, 1:1000, Dako; HNF4-a, 1:1000, Santa Cruz) overnight at 4 degrees and then switched to secondary antibodies (Alexa Flour 488 goat anti-rabbit, 1:1000, Invitrogen; Alexa Flour 594 rabbit anti-mouse, 1:2000, Invitrogen). Images were taken using Bio-Rad Zoe™ Fluorescent Cell Imager (Bio-Rad). For intracellular APOB localization, iPSC-heps were treated with 0, 3, 12, 48 μg/mL of DL-9 for 24 hours, and cells were fixed in 4% PFA followed by 0.1% Triton-X-100 penetration. Cells were then incubated with primary antibodies (anti-APOB 1:100, Mabtech; anti-LAMP1, 1:100, Cell Signaling, anti-SORTILIN, 1:100, Abcam) overnight at 4 degrees and switched to secondary antibodies as mentioned above. Images were taken using KEYENCE BZ-800 and Leica SP8 confocal microscopy. Integrated fluorescent intensity was normalized by total cell counts, which is determined by DAPI staining.

Cell Viability Assay

Cell viability assay was performed using CellTiter-Glo® luminescent cell viability assay kit following the manufacturer's instructions (Promega, Wis., US). Experimental and control groups were processed identically.

Quantitative Real-Time PCR Analysis

Human iPSC-derived hepatocytes (Day 20) were treated with 2 μg/mL of DL-1 for 24 hours. RNA was isolated from drug-treated and control iPSC-derived hepatocytes using the RNeasy Mini Kit (Qiagen, #74106). Genomic DNA was removed using the TURBO DNA-freeυ Kit (ThermoFisher, #AM1907). First-strand cDNA was synthesized using M-MLV Reverse Transcriptase (ThermoFisher, #28025-013). Quantitative real-time PCR was performed on a BioRad CFX384 real-time PCR machine.

Neutral Lipid Staining

K3 iPSC-derived hepatocytes (Day 20) were treated with or without 2 μg/mL of DL-1 or 20 nM of CP-346086 (Sigma) for 72 hours with daily medium change. At the time-point of interest, cells were treated with 2 μM of BODIPY® 493/503 staining solution (ThermoFisher) and 5 μg/mL of Hoechst 33342 (ThermoFisher) in PBS at 37 degrees for 15 minutes. Cells were washed with sterile PBS three times and then fixed with 4% PFA. Fluorescence images were taken using Bio-rad Zoeυ Fluorescent Cell Imager (Bio-Rad) and KEYENCE BZ-X800E Fluorescence Microscope (KEYENCE). Integrated fluorescent intensity was normalized by total cell counts, which is determined by DAPI staining.

Cholesterol Quantification

Cell Culture medium was harvested from human iPSC-derived hepatocytes (Day 20) before and after 24 hours of DL-1 (2 μg/mL) or vehicle treatment. Pre-drug and Post-drug medium were concentrated using Amico® Ultra 10K Centrifugal Filters (Sigma-Aldrich, #Z740171), and total cholesterol levels were determined using the colorimetric cholesterol assay kit (Sigma-Aldrich, #MAK043) following the manufactural instructions.

### REFERENCES

Si-Tayeb K, Noto F K, Sepac A, Sedlic F, Bosnjak Z J, Lough J W, Duncan S A: Generation of human induced pluripotent stem cells by simple transient transfection of plasmid DNA encoding reprogramming factors. BMC Dev Biol 2010, 10:81.
Mallanna S K, Duncan S A: Differentiation of hepatocytes from pluripotent stem cells. Curr Protoc Stem Cell Biol 2013, 26:1G 4 1-1G 4 13.
Liu J T, Lamprecht M P, Duncan S A: Using Human Induced Pluripotent Stem Cell-derived Hepatocyte-like Cells for Drug Discovery. J Vis Exp 2018(135).
Colella A D, Chegenii N, Tea M N, Gibbins I L, Williams K A, Chataway T K: Comparison of Stain-Free gels with traditional immunoblot loading control methodology. Anal Biochem 2012, 430(2):108-110.
Lee, H-J; Sarwade, B. D.; Park, J.; Kim, E. Synthesis of new photopolymeric methacrylate thioester with s-triazine ring for holographic recording. Optical Materials 2007, 30(4), 637-644.
Um, S.-I.; Lee, J.-K.; Kang, Y.; Baek, D.-J. The synthesis and properties of triazine-stilbene fluorescent brighteners containing the phenolic antioxidant. Dyes and Pigments 2004, 64(2), 93-99.
Pallavicini, M.; Fumagalli, L.; Gobbi, M.; Bolchi, C.; Colleoni, S.; Moroni, B.; Pedretti, A.; Rusconi, C.; Vistoli, G.; Valoti, E. QSAR study for a novel series of ortho disubstituted phenoxy analogues of al-adrenoceptor antagonist WB4101. European Journal of Medicinal Chemistry 2006, 41(9), 1025-1040.
Ortega, J. A.; Riccardi, L. M., Elirosa; B., Marco; Arencibia, J. M.; Greco, M. L.; Minarini, A.; Sissi, C.; De Vivo, M. Pharmacophore Hybridization. To Discover Novel Topoisomerase II Poisons with Promising Antiproliferative Activity. Journal of Medicinal Chemistry 2018, 61(3), 1375-1379.
Bergquist, B. L.; Jefferson, K. G.; Kintz, H. N.; Barber, A. E.; Yeagley, A. A. Disconnecting the Estrogen Receptor Binding Properties and Antimicrobial Properties of Parabens through 3,5-Substitution. ACS Medicinal Chemistry Letters 2018, 9(1), 51-55.

The invention is further described in the following numbered paragraphs:

1. A compound of formula (I):

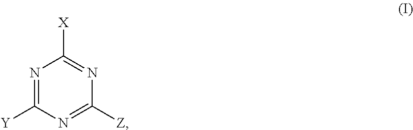

wherein:

X and Y, independently of each other, is halogen, SH, SC(O)CH₃, NH-lower alkyl, N(lower alkyl)₂, NH-alkoxy or NHNH₂;

Z is hydroxy, halogen, heterocycloalkyl or NR¹R²; and

R¹ and R², independently of each other, is hydrogen, alkyl, alkoxy, cycloalkyl, unsubstituted phenyl or phenyl mono-, bi- or tri-substituted independently with NH-phenyl, halogen, hydroxyl, lower alkyl or NH-heteroaryl, wherein said heteroaryl is unsubstituted or mono- or bi-substituted independently with SH or NHCH(CH₃)₂,

or a pharmaceutically acceptable salt thereof.

2. The compound according to paragraph 1, wherein X is SH.

3. The compound according to paragraph 1, wherein both X and Y are SH.

4. The compound according to paragraph 1, wherein both X and Y are SC(O)CH₃.

5. The compound according to any one of paragraphs 1-4, wherein Z is hydroxy, halogen or heterocycloalkyl.

6. The compound according to any one of paragraphs 1-5, wherein Z is NR¹R².

7. The compound according to paragraph 6, wherein R¹ and R2, independently of each other, is hydrogen, alkylor cycloalkyl.

8. The compound according to any one of paragraphs 6-7, wherein R¹ and R2, independently of each other, is unsubstituted phenyl.

9. The compound according to any one of paragraphs 6-8, wherein R¹ and R2, independently of each other, is phenyl mono-, bi- or tri-substituted independently with NH-phenyl, halogen, hydroxyl, lower alkyl or NH-heteroaryl, wherein said heteroaryl is unsubstituted or mono- or bi-substituted independently with SH or NHCH(CH₃)₂.

10. The compound according to any one of paragraphs 1-5, wherein Z is selected from

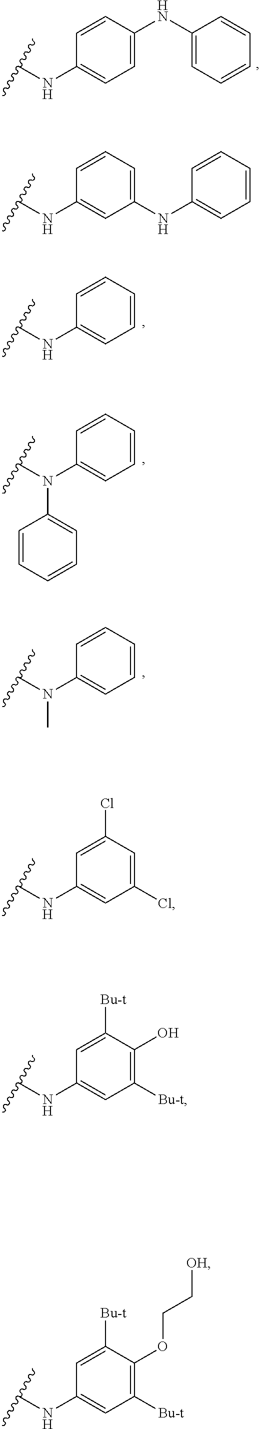

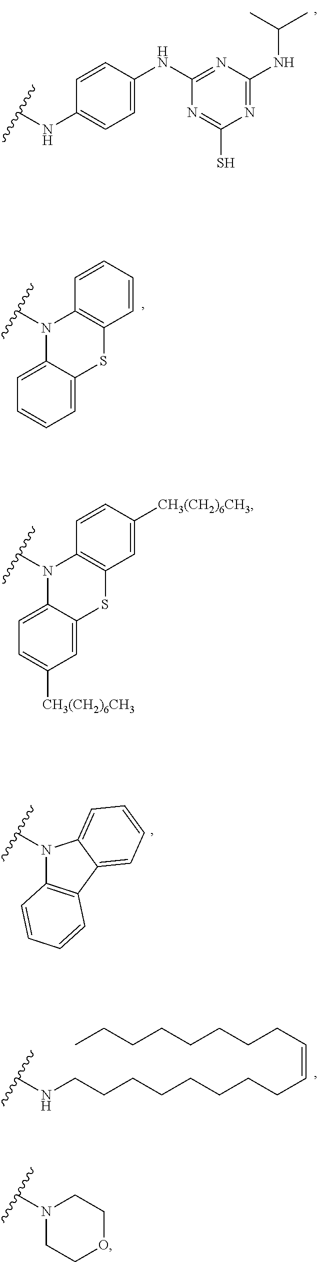

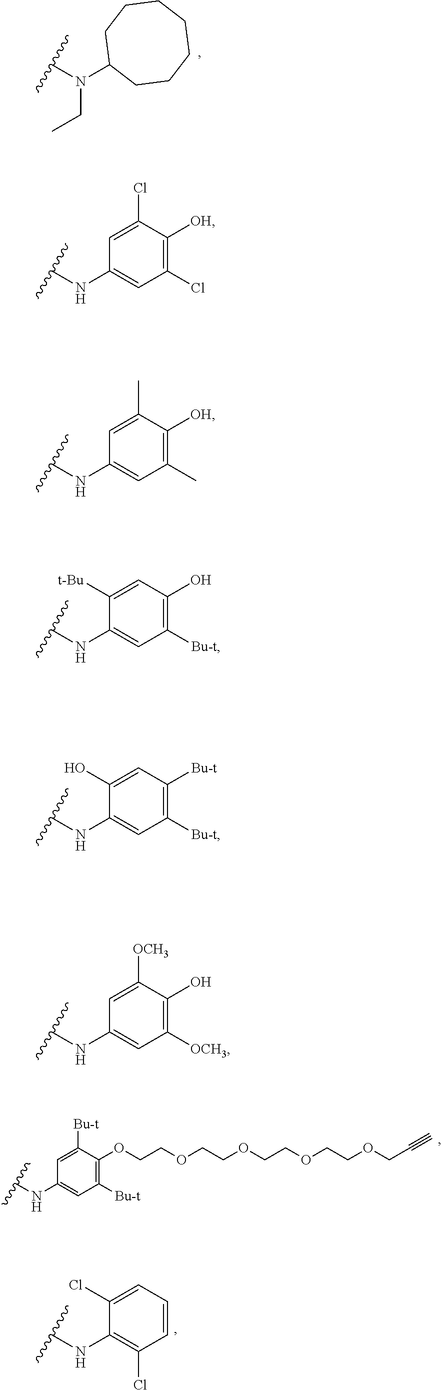

—NHCH₂CH₃, —N(CH₃)₂, NH-phenyl, —N(CH₂CH₃)₂, —N(CH(CH₃)₂)₂, —NHCH₃(CH₂)₆CH₃, —NHCH₃(CH₂)₁₀CH₃, and —NHCH₃(CH₂)₁₆CH₃.

11. The compound according to any one of paragraphs 1-10, wherein at least one of X and Y, is —NHCH₂CH₃, —N(CH₂CH₃)₂, NHCH(CH₃)₂, —N(CH₃)₂, or NHCH₂CH₂CH₂OCH₃.

12. The compound according to any one of paragraphs 1-11, wherein Y is NH-lower alkyl or N(lower alkyl)₂.

13. The compound according to paragraph 1, wherein said compound is:
N¹-(4,6-dichloro-1,3,5-triazin-2-yl)-N⁴-phenylbenzene-1,4-diamine;
S,S′-(6-((4-(phenylamino)phenyl)amino)-1,3,5-triazine-2,4-diyl) diethanethioate;
6-((4-(phenylamino)phenyl)amino)-1,3,5-triazine-2,4-dithiol;
S,S′-(6-((3-methoxypropyl)amino)-1,3,5-triazine-2,4-diyl)diethanethioate;
6-(phenylamino)-1,3,5-triazine-2,4-dithiol; or
N1-(4,6-dichloro-1,3,5-triazin-2-yl)-N4-phenylbenzene-1,4-diamine,
or a pharmaceutically acceptable salt thereof.

14. The compound according to paragraph 1, wherein said compound is:
6-{[4-(phenylamino)phenyl]amino}-1,3,5-triazine-2,4-dithiol;
[4-(phenylamino)-6-sulfanidyl-1,3,5-triazin-2-yl]sulfanide;
6-(diphenylamino)-1,3,5-triazine-2,4-dithiol;
6-[methyl(phenyl)amino]-1,3,5-triazine-2,4-dithiol;
6-[(3,5-dichlorophenyl)amino]-1,3,5-triazine-2,4-dithiol;
6-[(2-chlorophenyl)amino]-1,3,5-triazine-2,4-dithiol;
4-(ethylamino)-6-{[4-(phenylamino)phenyl]amino}-1,3,5-triazine-2-thiol;
4-(diethylamino)-6-{[4-(phenylamino)phenyl]amino}-1,3,5-triazine-2-thiol;
{4-[(3,5-di-tert-butyl-4-hydroxyphenyl)amino]-6-sulfanidyl-1,3,5-triazin-2-yl}sulfanide;
4-[(propan-2-yl)amino]-6-{[4-({4-[(propan-2-yl)amino]-6-sulfanyl-1,3,5-triazin-2-
yl}amino)phenyl]amino}-1,3,5-triazine-2-thiol;
6-(ethylamino)-1,3,5-triazine-2,4-dithiol;
6-(dimethylamino)-1,3,5-triazine-2,4-dithiol;
4-[(3-methoxypropyl)amino]-6-(phenylamino)-1,3,5-triazine-2-thiol;
[4-(diethylamino)-6-sulfanidyl-1,3,5-triazin-2-yl]sulfanide;
6-[bis(propan-2-yl)amino]-1,3,5-triazine-2,4-dithiol;
6-(octylamino)-1,3,5-triazine-2,4-dithiol;
6-(dodecylamino)-1,3,5-triazine-2,4-dithiol;
6-(octadecylamino)-1,3,5-triazine-2,4-dithiol;
6-{[(9Z)-octadec-9-en-1-yl]amino}-1,3,5-triazine-2,4-dithiol;
6-(morpholin-4-yl)-1,3,5-triazine-2,4-dithiol;
6-[cyclooctyl(ethyl)amino]-1,3,5-triazine-2,4-dithiol;
6-(3,5-di-tert-butyl-4-(2-hydroxyethoxy)-phenylamino)-1,3,5-triazine-2,4-dithiol;
6-(3,5-dichloro-4-hydroxyphenylamino)-1,3,5-triazine-2,4-dithiol;
6-(3,5-dimethyl-4-hydroxyphenylamino)-1,3,5-triazine-2,4-dithiol;
6-(2,4-di-tert-butyl-5-hydroxyphenylamino)-1,3,5-triazine-2,4-dithiol;
6-(3,5-di-tert-butyl-2-hydroxyphenylamino)-1,3,5-triazine-2,4-dithiol;
6-(3,5-di-tert-butyl-4-(2-(2-(2-(2-(2-propyn-1-yloxy)ethoxy)ethyoxy)etoxy)ethoxy)-phenylamino)-1,3,5-triazine-2,4-dithiol; or
6-(3,5-di-tert-butyl-4-(2-(2-(2-(2-(2-propyn-1-yloxy)ethoxy)ethyoxy)etoxy)ethoxy)-phenylamino)-1,3,5-triazine-2,4-dithiol,
or a pharmaceutically acceptable salt thereof

15. A pharmaceutical composition, comprising a therapeutically effective amount of a compound according to any one of to any one of paragraphs 1-14, or a pharmaceutically acceptable salt thereof, and a pharmaceutically acceptable carrier.

16. A method for the treatment of hypercholesterolemia, comprising the step of administering to a patient in need thereof a therapeutically effective amount of a compound according to any one of paragraphs 1-14, or a pharmaceutically acceptable salt thereof, and a pharmaceutically acceptable carrier.

It is to be understood that the invention is not limited to the particular embodiments of the invention described above, as variations of the particular embodiments may be made and still fall within the scope of the appended claims.

1. A compound of formula (I): 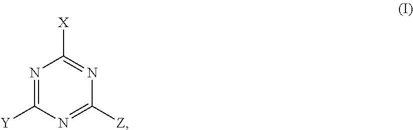
wherein:
X and Y, independently of each other, is halogen, SH, SC(O)CH₃, NH-lower alkyl, N(lower alkyl)₂, NH-alkoxy or NHNH₂;
Z is hydroxy, halogen, heterocycloalkyl or NR¹R²; and
R¹ and R², independently of each other, is hydrogen, alkyl, alkoxy, cycloalkyl, unsubstituted phenyl or phenyl mono-, bi- or tri-substituted independently with NH-phenyl, halogen, hydroxyl, lower alkyl or NH-heteroaryl, wherein said heteroaryl is unsubstituted or mono- or bi-substituted independently with SH or NHCH(CH₃)₂,
or a pharmaceutically acceptable salt thereof.

2. The compound according to claim 1, wherein X is SH.

3. The compound according to claim 1, wherein both X and Y are SH.

4. The compound according to claim 1, wherein both X and Y are SC(O)CH₃.

5. The compound according to claim 1, wherein Z is hydroxy, halogen or heterocycloalkyl.

6. The compound according to claim 1, wherein Z is NR¹R².

7. The compound according to claim 1, wherein R¹ and R², independently of each other, is hydrogen, alkylor cycloalkyl.

8. The compound according to claim 1, wherein R¹ and R², independently of each other, is unsubstituted phenyl.

9. The compound according to claim 1, wherein R¹ and R², independently of each other, is phenyl mono-, bi- or tri-substituted independently with NH-phenyl, halogen, hydroxyl, lower alkyl or NH-heteroaryl, wherein said heteroaryl is unsubstituted or mono- or bi-substituted independently with SH or NHCH(CH₃)₂.

10. The compound according to claim 1, wherein Z is selected from 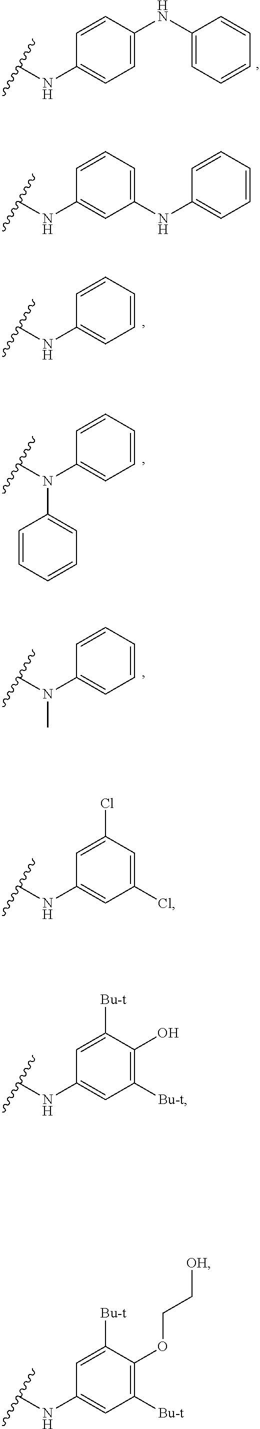 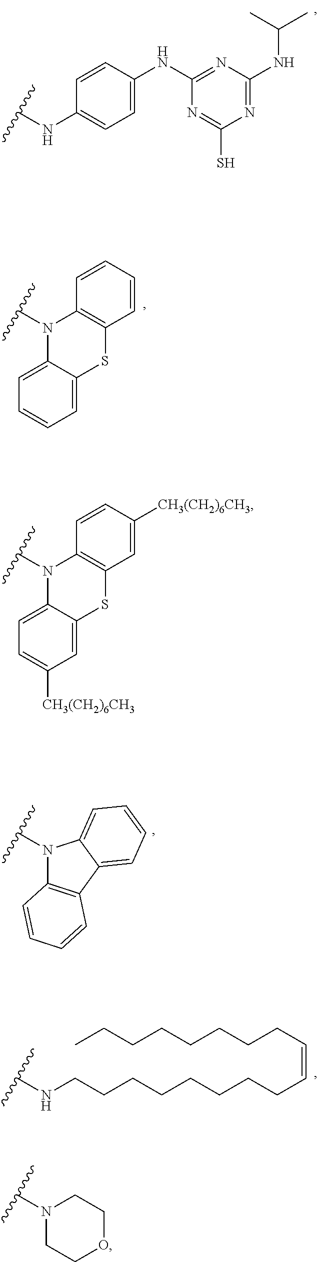 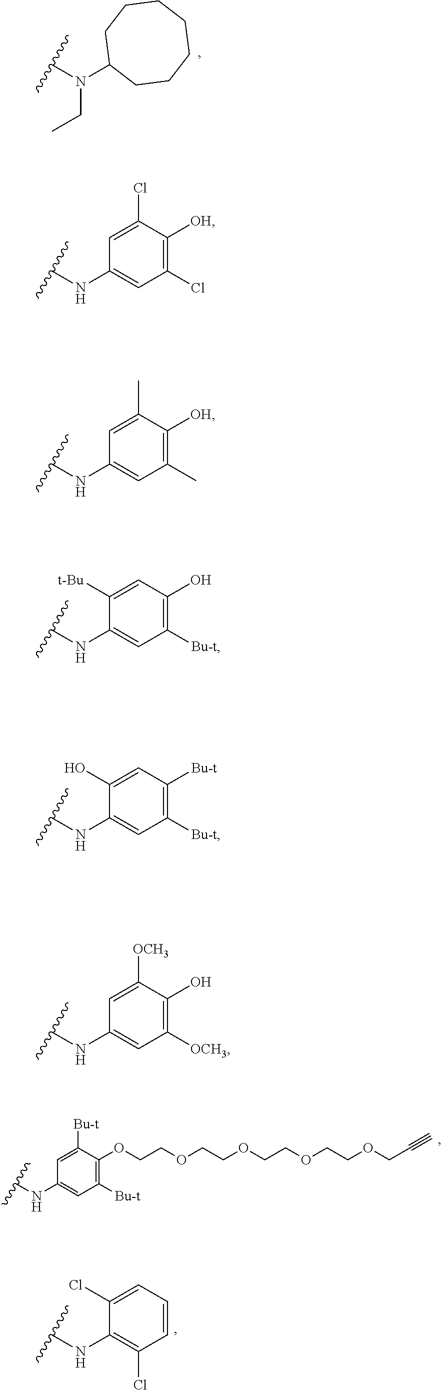
—NHCH₂CH₃, —N(CH₃)₂, NH-phenyl, —N(CH₂CH₃)₂, —N(CH(CH₃)₂)₂, —NHCH₃(CH₂)₆CH₃, —NHCH₃(CH₂)₁₀CH₃, and —NHCH₃(CH₂)₁₆CH₃.

11. The compound according to claim 1, wherein at least one of X and Y, is —NHCH₂CH₃, —N(CH₂CH₃)₂, NHCH(CH₃)₂, —N(CH₃)₂, or NHCH₂CH₂CH₂OCH₃.

12. The compound according to claim 1, wherein Y is NH-lower alkyl or N(lower alkyl)₂.

13. The compound according to claim 1, wherein said compound is:
N¹-(4,6-dichloro-1,3,5-triazin-2-yl)-N⁴-phenylbenzene-1,4-diamine;
S,S′-(6-((4-(phenylamino)phenyl)amino)-1,3,5-triazine-2,4-diyl) diethanethioate;
6-((4-(phenylamino)phenyl)amino)-1,3,5-triazine-2,4-dithiol;
S,S′-(6-((3-methoxypropyl)amino)-1,3,5-triazine-2,4-diyl)diethanethioate;
6-(phenylamino)-1,3,5-triazine-2,4-dithiol; or
N1-(4,6-dichloro-1,3,5-triazin-2-yl)-N4-phenylbenzene-1,4-diamine,
or a pharmaceutically acceptable salt thereof.

14. The compound according to claim 1, wherein said compound is:
6-{[4-(phenylamino)phenyl]amino}-1,3,5-triazine-2,4-dithiol;
[4-(phenylamino)-6-sulfanidyl-1,3,5-triazin-2-yl]sulfanide;
6-(diphenylamino)-1,3,5-triazine-2,4-dithiol;
6-[methyl(phenyl)amino]-1,3,5-triazine-2,4-dithiol;
6-[(3,5-dichlorophenyl)amino]-1,3,5-triazine-2,4-dithiol;
6-[(2-chlorophenyl)amino]-1,3,5-triazine-2,4-dithiol;
4-(ethylamino)-6-{[4-(phenylamino)phenyl]amino}-1,3,5-triazine-2-thiol;
4-(diethylamino)-6-{[4-(phenylamino)phenyl]amino}-1,3,5-triazine-2-thiol;
{4-[(3,5-di-tert-butyl-4-hydroxyphenyl)amino]-6-sulfanidyl-1,3,5-triazin-2-yl}sulfanide;
4-[(propan-2-yl)amino]-6-{[4-({4-[(propan-2-yl)amino]-6-sulfanyl-1,3,5-triazin-2-yl}amino)phenyl]amino}-1,3,5-triazine-2-thiol;
6-(ethylamino)-1,3,5-triazine-2,4-dithiol;
6-(dimethylamino)-1,3,5-triazine-2,4-dithiol;
4-[(3-methoxypropyl)amino]-6-(phenylamino)-1,3,5-triazine-2-thiol;
[4-(diethylamino)-6-sulfanidyl-1,3,5-triazin-2-yl]sulfanide;
6-[bis(propan-2-yl)amino]-1,3,5-triazine-2,4-dithiol;
6-(octylamino)-1,3,5-triazine-2,4-dithiol;
6-(dodecylamino)-1,3,5-triazine-2,4-dithiol;
6-(octadecylamino)-1,3,5-triazine-2,4-dithiol;
6-{[(9Z)-octadec-9-en-1-yl]amino}-1,3,5-triazine-2,4-dithiol;
6-(morpholin-4-yl)-1,3,5-triazine-2,4-dithiol;
6-[cyclooctyl(ethyl)amino]-1,3,5-triazine-2,4-dithiol;
6-(3,5-di-tert-butyl-4-(2-hydroxyethoxy)-phenylamino)-1,3,5-triazine-2,4-dithiol;
6-(3,5-dichloro-4-hydroxyphenylamino)-1,3,5-triazine-2,4-dithiol;
6-(3,5-dimethyl-4-hydroxyphenylamino)-1,3,5-triazine-2,4-dithiol;
6-(2,4-di-tert-butyl-5-hydroxyphenylamino)-1,3,5-triazine-2,4-dithiol;
6-(3,5-di-tert-butyl-2-hydroxyphenylamino)-1,3,5-triazine-2,4-dithiol;
6-(3,5-di-tert-butyl-4-(2-(2-(2-(2-(2-propyn-1-yloxy)ethoxy)ethyoxy)etoxy)ethoxy)-phenylamino)-1,3,5-triazine-2,4-dithiol; or
6-(3,5-di-tert-butyl-4-(2-(2-(2-(2-(2-propyn-1-yloxy)ethoxy)ethyoxy)etoxy)ethoxy)-phenylamino)-1,3,5-triazine-2,4-dithiol,
or a pharmaceutically acceptable salt thereof

15. A pharmaceutical composition, comprising a therapeutically effective amount of a compound according to claim 1, or a pharmaceutically acceptable salt thereof, and a pharmaceutically acceptable carrier.

16. A method for the treatment of hypercholesterolemia, comprising the step of administering to a patient in need thereof a therapeutically effective amount of a compound according to claim 1, or a pharmaceutically acceptable salt thereof, and a pharmaceutically acceptable carrier.
